(19) United States
(12) Patent Application Publication
(10) Pub. No.: US 2024/0425930 A1
(43) Pub. Date: Dec. 26, 2024

(54) METHODS FOR SELECTIVE SEQUENCING OF CANCER DNA

(71) Applicant: Inivata Ltd., Cambridge (GB)

(72) Inventors: Tim Forshew, Stevanage (GB); Robert Osborne, Cambridge (GB); Nitzan Rosenfeld, Cambridge (GB); Uri David Akavia, Cambridge (GB)

(73) Assignee: Inivata Ltd., Cambridge (GB)

(21) Appl. No.: 18/747,597

(22) Filed: Jun. 19, 2024

Related U.S. Application Data
(60) Provisional application No. 63/509,603, filed on Jun. 22, 2023.
(60) Provisional application No. 63/609,943, filed on Dec. 14, 2023.

(51) Int. Cl. C12Q 1/6886; C12N 15/10; C12Q 1/6806; C12Q 1/686; C12Q 1/6874

(52) U.S. Cl. CPC C12Q 1/6886; C12N 15/1065; C12Q 1/6806; C12Q 1/686; C12Q 1/6874; C12Q 2600/112

(57) ABSTRACT

A method for selective sequencing of a plurality of target regions from a patient is provided. A pool of oligonucleotides comprises a first sub-population targeting the plurality of target regions and a second sub-population. Each member of the first sub-population of oligonucleotides comprises a first sequence that is complementary to one of the plurality of target regions from the patient and an identifier sequence specific to the patient, and each member of the second sub-population of oligonucleotides comprises a first sequence that is complementary to one of the plurality of target regions from a second patient and an identifier sequence specific to the second patient. A test sample from the patient is contacted with the pool, and then contacted with oligonucleotides comprising a sequence that is complementary to the identifier sequence specific to the first patient. The plurality of target regions from the patient are then selected and sequenced.

### CROSS-REFERENCING

This application claims the benefit of U.S. provisional application Ser. No. 63/609,943, filed on Dec. 14, 2023, and U.S. provisional application Ser. No. 63/509,603, filed on Jun. 22, 2023; the contents of which are hereby incorporated by reference in their entirety.

### FIELD

The present disclosure relates to methods for selective sequencing of a plurality of target regions from a patient, each of the target regions containing, or suspected of containing, one or more cancer specific variants.

### BACKGROUND

In many cases, cancer treatment may require at least two steps: a first treatment intended to remove the tumor cells then a second treatment aiming to eradicate any remaining cancer cells in the patient's body if the initial treatment is not completely successful. The treatment used to eradicate the remaining cancer cells often differs from the first treatment.

The small number of cancer cells that remain in the person after initial treatment when a patient may apparently be in remission is often called “minimal residual disease” (MRD) or residual disease and may be the cause of relapse in many cancers. It is critical to determine the likelihood of a patient having disease recurrence and relapsing following initial treatment so that those most likely to need additional treatment can receive additional treatment, while those that don't need additional treatment are spared, thereby reducing harm to the patient and decreasing the cost of treatment. It is also critical to have sensitive methods that detect risks of cancer recurrence earlier than current methods (e.g., which are usually done by imaging or clinical analysis). For example, existing imaging methods may struggle to detect residual tumors of less than 1 c³ or may not be able to distinguish residual disease from pseudo-progression.

MRD has been successfully detected in some hematological malignancies where relatively large amounts of DNA can be analyzed. MRD can also be detected for many solid tumors by assessing cell free DNA (cfDNA) for circulating tumor DNA (ctDNA). The problem with detecting minimal residual disease in cfDNA, however, is that many of the tests used to detect sequence variations in a sample are not sensitive enough. Specifically, the frequency at which an individual tumor sequence variation is expected to occur in the cfDNA of patients that have minimal residual disease is typically well below the frequency at which sequencing artefacts are generated by PCR errors, base mis-calls and/or DNA damage. This problem is compounded by the fact that, in some cases, the level of mutant DNA may be so low that, on average, there is less than a single copy of each mutation being assessed in the cfDNA sample being analyzed. Thus, detection of minimal residual disease by sequencing-based approaches has remained challenging.

Some highly sensitive sequencing methods for detecting cancer DNA do exist, and can be used to diagnose minimal residual disease, among other things. Such methods typically use next-generation sequencing (NGS) platforms and incorporate built-in controls and error-correction for highly sensitive and specific variant detection. Such methods are also typically personalized assays that can track a set of multiple tumor specific variants in a patient using a liquid biopsy with exceptional sensitivity, allowing both detection of residual disease following curative intent or definitive treatment and early detection of relapse.

These existing sequencing methods may have certain advantages over conventional methods such as imaging. For example, the method may be used to consistently and reliably determine whether a DNA sample has cancer DNA, even if the fraction of cancer DNA in the sample is less than 0.01%. This is well below the level of sensitivity of conventional methods, and well below the frequencies at which sequencing artefacts can be generated by errors. By assessing several sequence variations (e.g. by tracking a set of 48 tumor-specific variants in a patient) these methods are also able to detect cancer DNA in a sample of DNA in which there is on average less than a single copy of each individual sequence variation. Since the assays are personalized, the patient specific primer panel targeting the up to multiple (e.g. 48) variants is different for each patient. Synthesizing these personalized oligonucleotide panels is therefore a sunk cost that must be paid for each patient.

There is still a need in the art for additional sensitivity gain, as even highly sensitive methods miss cases of MRD, making such a gain clinically valuable. In order to increase the sensitivity 50× to 1000×, the number of variants targeted per patient needs to increase significantly. However, the cost of synthesizing patient specific oligonucleotide primer panels to accommodate the larger number of variants is currently a limiting factor in scaling up the existing methods. Assuming preparation of a patient specific primer panel costs on average £400-£500 for 48 variants, scaling the assay up 100-fold means the cost for a patient specific primer panel for 4800 variants is £40,000-50,000 per patient, which is prohibitive.

There therefore remains in the art a need for detecting MRD in an extremely sensitive yet cost efficient manner.

In the future, the costs associated with targeted sequencing-including costs of synthesizing oligonucleotide primer panels targeting certain variants-are likely to fall as technologies advance. It may therefore become possible to utilize very large oligonucleotide pools. There is therefore also a need in the art for sensitive methods of detecting MRD that can leverage very large oligonucleotide pools as they become financially viable.

### SUMMARY

The present disclosure provides methods for selective sequencing of a plurality of target regions, preferably from a patient, each of the target regions containing, or suspected of containing, one or more genetic variants, such as cancer specific variants. These methods allow methods of detecting MRD to be scaled up to dramatically increase their sensitivity in a cost-efficient manner. The methods facilitate use of a large pool of oligonucleotides which can be utilized for multiple patients, as opposed to synthesis of a small set of oligonucleotides for each patient. Subsets of the large oligonucleotide pool can be used to target specific regions of interest, allowing efficiencies of scale in terms of oligonucleotide synthesis to be applied to personalized assays.

The methods of the present disclosure can be applied and combined with existing methods of detecting cancer DNA in a sample, for example, the methods shown in FIG. 1 and those disclosed in commonly owned International Patent Publication WO 2023/012521. Specifically, any of the methods for selective sequencing disclosed herein may additionally comprise steps of: (a) sequencing multiple aliquots of the test sample to produce, for each aliquot, sequence reads corresponding to two or more target regions that each have one or more sequence variations present within the patient's cancer; (b) for each aliquot, for each target region: i. determining the number of sequence reads that have the sequence variation; ii. determining the total number of sequence reads; and iii. comparing i. and ii. to one or more error probability distribution models for the sequence variation, wherein the one or more models are obtained from DNA that does not contain the sequence variation; and (c) integrating the collective results of step (b) to determine if there is cancer DNA in the test sample.

In a first aspect, provided are methods for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. contacting a test sample from the first patient with the pool of oligonucleotides;
c. contacting the test sample with oligonucleotides each comprising a sequence that is complementary to the identifier sequence specific to the first patient;
d. selectively sequencing the plurality of target regions from the first patient optionally thereby identifying the presence or absence of the cancer specific variants.

In some embodiments there is an amplification step between steps b. and c. to produce an amplified test sample. Step c. may be equally performed on the amplified test sample.

In a second aspect, provided are methods for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of primer oligonucleotides, wherein each member of the first sub-population of primer oligonucleotides comprises a first sequence that is complementary to one of the target regions from the first patient and an identifier sequence specific to the first patient;
   ii. a second sub-population of primer oligonucleotides, wherein each member of the second sub-population of primer oligonucleotides comprises a first sequence that is complementary to one of a plurality of target regions from a second patient and an identifier sequence specific to the second patient;
b. contacting a test sample from the first patient with the pool of oligonucleotides;
c. performing PCR on the test sample comprising the pool of oligonucleotides to produce an amplified test sample;
d. enriching the amplified test sample for amplicons derived from the first patient's target regions by contacting the amplified test sample with patient-specific primer pairs and performing PCR, wherein one or both primers comprises a sequence that is complementary to a identifier sequence specific to the first patient present in the first sub-population of oligonucleotides from step (a);
e. selectively sequencing the enriched sample to determine the sequence of the plurality of target regions from the first patient, optionally thereby identifying the presence or absence of the cancer specific variants.

In some embodiments, the first sub-population of primer oligonucleotides comprise common sequences that are selectively targeted by the patient-specific primer pairs. The primer oligonucleotides may be pairs of forward and reverse primers targeting the plurality of target regions. In some embodiments, the primer oligonucleotides are forward or reverse primers targeting the plurality of target regions but they are not paired and a universal sequence is ligated to the test sample, and the PCR utilizes the forward or reverse target specific primer and a common primer targeting the ligated universal sequence.

In some embodiments, the patient-specific primers further comprise sequencing adaptors. The sequencing adaptors may provide compatibility with a particular sequencing platform. The sequencing platform may be an Illumina sequencing platform, a PacBio Onso sequencing platform, a Element sequencing platform or an Ultima sequencing platform, among others.

In a third aspect, provided are methods for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of bait oligonucleotides, wherein the first sub-population of oligonucleotides comprises oligonucleotide baits that target the plurality of target regions from the first patient, wherein each member of the first sub-population of oligonucleotides comprises a first sequence that is complementary to one of the target regions from the first patient and an identifier sequence specific to the first patient;
   ii. a second sub-population of bait oligonucleotides, wherein the second sub-population of oligonucleotides comprises oligonucleotide baits wherein each member of the second sub-population of oligonucleotides comprises a first sequence that is complementary to one of a plurality of target regions from a second patient and an identifier sequence specific to the second patient;
b. contacting a test sample from the first patient or an amplified test sample derived from the test sample with the pool of oligonucleotides;
c. contacting the test sample with patient-specific oligonucleotides each comprising a binding moiety and a sequence that is complementary to the identifier sequence specific to the first patient, to produce a tagged test sample;
d. conducting a binding moiety pull down assay on the tagged test sample to obtain an enriched sample enriched for amplicons derived from the first patient's target regions;
e. selectively sequencing the enriched sample to determine the sequence of the plurality of target regions from the first patient, optionally thereby identifying the presence or absence of the cancer specific variants.

In some embodiments, the method further comprises a step of performing PCR on the test sample from the first patient after step (a) to produce an amplified test sample.

In some embodiments, the binding moiety is a biotin. In some embodiments the binding moiety is attached to a support. The support may be a streptavidin bead or other suitable support. The method may further comprise making a sequencing library from the test sample using any suitable method known in the art. For example, a Y-stem library may comprise one or more nucleic acid adaptors comprising two nucleic acid strands, wherein the strands are complementary to each other at one end but not the other. The Y-stem adaptor is therefore double stranded only at one end. Other approaches may include using hairpin adaptors or universal half-functional adapters, such as those used by New England Biolabs or ArcherDx, respectively.

In a fourth aspect, provided are methods for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of bridge oligonucleotides, wherein the first sub-population of oligonucleotides comprises at least two classes of bridge probe that target the plurality of target regions from the first patient, wherein the first class of bridge probe comprises:
      1. a first sequence that is complementary to one of the target regions from the first patient; and
      2. An identifier sequence specific to the first patient, wherein the identifier sequence is a first adaptor landing sequence capable of hybridizing to a first bridge binding sequence of an anchor probe specific to the first patient,
         and wherein the second class of bridge probe comprises:
      1. a first sequence that is specific to same target region from the first patient as is targeted by the first class of bridge probe but targeting a sequence that flanks (upstream or downstream) the sequence in the target region targeted by the first class of bridge probe; and
      2. an identifier sequence specific to the first patient, wherein the identifier sequence is a second adaptor landing sequence capable of hybridizing to a second bridge binding sequence of the anchor probe specific to the first patient;
   ii. a second sub-population of bridge oligonucleotides, wherein the second sub-population of oligonucleotides comprises at least two classes of bridge probes wherein the first class of bridge probe comprises:
      1. a first sequence that is complementary to one of a plurality of target regions from a second patient; and
      2. An identifier sequence specific to the second patient, wherein the identifier sequence is an adaptor landing sequence capable of hybridizing to a first bridge binding sequence of an anchor probe specific to the second patient,
         and wherein the second class of bridge probe comprises:
      1. a first sequence that is specific to the same target region from the second patient as is targeted by the first class of bridge probe but targeting a sequence that flanks (upstream or downstream) the sequence in the target region targeted by the first class of bridge probe; and
      2. an identifier sequence specific to the second patient, wherein the identifier sequence is an adaptor landing sequence capable of hybridizing to a second bridge binding sequence of the anchor probe specific to the second patient;
b. contacting a test sample from the first patient with the pool of oligonucleotides;
c. enriching the test sample for nucleic acid molecules from or derived from the first patient using a hybridization capture method by contacting the test sample with patient-specific oligonucleotides, wherein the patient specific oligonucleotides are patient specific anchor probes each comprising a first bridge binding sequence and a second bridge binding sequence that are complementary to the first and second adaptor landing sequences of the patient-specific sequence specific to the first patient;
d. selectively sequencing the enriched sample to determine the sequence of the plurality of target regions from the first patient.

In some embodiments there is an amplification step between steps b. and c. to produce an amplified test sample. Step c. may be equally performed on the amplified test sample.

In some embodiments, the patient specific anchor probes comprise a binding moiety. The binding moiety may be a biotin. It would be understood that all references to “biotin” and “biotinylation” herein may refer to free biotin, biotin bound to an oligonucleotide, or biotin forming part of a nucleotide that comprises biotinylated dNTPs.

In a fifth aspect, provided are methods for selective sequencing of a plurality of target regions, each of the target regions containing, or suspected of containing, one or more genetic variants, the method comprising:
a. providing a pool of oligonucleotides, wherein the pool of oligonucleotides comprises:
   i. a first sub-population of oligonucleotides targeting a first subset of the plurality of target regions, wherein each member of the first sub-population of oligonucleotides comprises a first sequence that is complementary to one of the first subset of the plurality of target regions and a first identifier sequence;
   ii. a second sub-population of oligonucleotides, wherein each member of the second sub-population of oligonucleotides comprises a first sequence that is complementary to one of second subset of the plurality of target regions and a second identifier sequence;
b. contacting a test sample from a first patient with the pool of oligonucleotides;
c. contacting the test sample with oligonucleotides each comprising a sequence that is complementary to the first identifier sequence;
d. selectively identifying the presence or absence of genetic variants in the first subset of the plurality of target regions in the test sample.

In some embodiments, the genetic variants are previously identified in a cancer sample from a first patient.

The methods may further comprise contacting the test sample with oligonucleotides each comprising a sequence that is complementary to the second identifier sequence, and selectively sequencing the second subset of the plurality of target regions, optionally thereby identifying the presence or absence of the genetic variants in the second subset of the plurality of target regions in the test sample.

In some embodiments the methods further comprise contacting a test sample from a second patient with the pool of oligonucleotides. In some embodiments the methods further comprise contacting the test sample from the second patient with oligonucleotides each comprising a sequence that is complementary to the second identifier sequence, and selectively identifying the presence or absence of genetic variants in the second subset of the plurality of target regions in the test sample.

In some embodiments, selectively identifying the presence or absence of the genetic variants comprises sequencing and/or amplifying the first subset of the plurality of target regions.

In some embodiments of any of the aspects described herein, the plurality of target regions from a first patient are genomic target regions. The number of target regions from the first and/or second patient may be at least about 100. The number of target regions from the first and/or second patient may be from about 100 to about 100,000 target regions. The number of target regions from the first and/or second patient may be from about 100 to about 50,000target regions. The number of target regions from the first and/or second patient may be at least about 1000. The number of target regions from the first and/or second patient may be from about 1000 to about 20,000 target regions. The number of target regions from the first and/or second patient may be at least about 3000. The number of target regions from the first and/or second patient may be from about 3000 to about 20,000 target regions. In some preferred embodiments, number of target regions from the first and/or second patient is from about 1000 to about 20,000 target regions.

In some embodiments of any of the aspects described herein, the one or more cancer specific variants were previously identified in a cancer from the first patient. The one or more cancer specific variants may be known cancer specific variants.

In some embodiments of any of the aspects described herein, the patient has or has previously had cancer.

In some embodiments of any of the aspects described herein, the method comprises, before step (a), identifying one or more cancer specific variants that are present within the patient's cancer. The methods may comprise identifying 100 or more cancer specific variants that are present within the patient's cancer, or identifying 1000 or more cancer specific variants that are present within the patient's cancer.

In some embodiments of any of the aspects described herein, the methods comprise sequencing of nucleic acid molecules from or derived from the first patient and does not comprise sequencing of nucleic acid molecules from or derived from the second (or any other) patient.

In some embodiments of any of the aspects described herein, the methods further comprise performing the same method for the second patient using a test sample from a second patient and patient-specific oligonucleotides specific to the second patient. For example, in some embodiments the methods may further comprise:
e. contacting a test sample from the second patient with the pool of oligonucleotides;
f. contacting the test sample with patient-specific oligonucleotides each comprising a sequence that is complementary to the patient-specific sequence specific to the second patient;
g. using the patient-specific oligonucleotides, selectively sequencing the plurality of target regions from the second patient, optionally thereby identifying the presence or absence of the cancer specific variants.

In some embodiments of any of the aspects described herein, each oligonucleotide in the first sub-population of oligonucleotides comprises the same patient-specific sequence specific to the first patient, or wherein each oligonucleotide in the first sub-population of oligonucleotides comprises either the same patient-specific sequence specific to the first patient or the reverse complement thereof. The pool of oligonucleotides may further comprise N additional sub-populations of oligonucleotides, each additional sub-population specific for another patient. In some embodiments, the pool of oligonucleotides further comprises N additional patient-specific sub-populations of oligonucleotides, wherein each member of each patient-specific sub-population of oligonucleotides comprises a first sequence that is complementary to one of a plurality of target regions from that sub-population's patient, and a patient-specific sequence specific to that sub-population's patient, wherein N is from 1 to 50, or N is from 1 to 200, or N is from 1 to 1000.

In a further aspect, a computer-readable storage medium or media storing instructions for performing the methods disclosed herein is provided.

In a further aspect, a method of diagnosing cancer in a patient is provided, comprising performing any of the methods described herein on a test sample obtained from the patient.

In a further aspect, a method of treating cancer in a patient is provided, comprising determining the presence or absence of cancer DNA in a test sample according to the methods described herein, and administering a cancer therapy or treatment to the patient, or recommending administration of a cancer therapy or treatment to the patient. In some embodiments the patient has been identified as still having cancer or suspected of having cancer following a treatment according to the methods disclosed herein.

In still further aspects are provide methods of determining the effectiveness of a cancer treatment or therapy, methods of monitoring the effect of a cancer therapy or treatment, or methods of detecting or monitoring minimal residual disease (MRD). In some embodiments the methods comprise administering the cancer treatment or therapy to a patient, obtaining a test sample from the patient, and determining the presence, absence or amount of cancer DNA in the test sample according to the methods for selective sequencing described herein. In some embodiments the methods comprise using test samples obtained from the patient at two or more time points during or after the administration of the cancer therapy or treatment. In some embodiments the methods comprise obtaining or having obtained a test sample from a patient that has undergone a cancer therapy or treatment, and performing a method of selective sequencing described herein.

### BRIEF DESCRIPTION OF THE FIGURES

The skilled artisan will understand that the drawings, described below, are for illustration purposes only. The drawings are not intended to limit the scope of the present teachings in any way. The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a flow diagram depicting an exemplary embodiment of a method for carrying out a personalized, tumor-informed assay for detecting cancer DNA.

FIG. 2A is a flow diagram depicting an exemplary embodiment of a method for designing a plurality of oligonucleotides for use with methods of selectively sequencing cancer DNA.

FIG. 2B is a flow diagram depicting an exemplary embodiment of a method for selective sequencing of target regions from a first patient, each of the target regions containing, or suspected of containing, one or more cancer specific variants.

FIG. 3A is a diagram depicting an exemplary embodiment of a method for selective sequencing using a PCR amplification technique.

FIG. 3B is a diagram depicting another exemplary embodiment of a method for selective sequencing using a PCR amplification technique, wherein the method includes using DNA with ligated adaptors as a template, a universal primer against the adaptor and the pool of oligonucleotides.

FIG. 4 is a diagram depicting an exemplary embodiment of a method for selective sequencing using a hybridization capture technique.

FIG. 5 is a diagram depicting another exemplary embodiment of a method for selective sequencing using a synergistic indirect DNA hybrid capture technique.

FIG. 6A is a diagram depicting another exemplary embodiment of a method for selective sequencing using a hybridization capture technique with an additional pool enrichment step.

FIG. 6B depicts an exemplary embodiment of a method for selective sequencing using a PCR amplification technique and biotinylated primers.

FIG. 7A depicts an example embodiment of a selective sequencing method using a hybridization technique and a DNA cleavage approach using double stranded oligonucleotides.

FIG. 7B depicts another example embodiment of a selective sequencing method using a hybridization technique wherein the DNA is bound to a solid support, and a DNA cleavage approach.

FIG. 7C depicts another example embodiment of a selective sequencing method using a hybridization technique, wherein the “bait” oligonucleotides are synthesized in parallel on a solid support.

### DETAILED DESCRIPTION

The inventors have recognized and appreciated that detection of cancer DNA in a test sample when using a tumour informed assay is limited by the number of DNA molecules (e.g., cfDNA) in a sample that are available for testing and the number of cancer mutations that are tested for in the sample. Increasing either factor will increase the sensitivity of the assay, allowing for the detection of cancer DNA even at a very low variant allele frequency (e.g., <0.01%, <0.001%, <0.0001%). The number of DNA molecules can be increased by using a high efficiency library preparation method such as multiplex PCR using short amplicons or ligation-based library preparation wherein the ligation and clean-up steps are optimized to convent a high percentage of the DNA into a readable library, which may enable up to 20,000 (and typically about 10,000) genome equivalents available for testing. Over 20,000 genome equivalents may be available for testing in some cases, depending on the source of the DNA sample. Additionally, the cost of sequencing is rapidly dropping, making it practical from a sequencing perspective to increase the number of cancer mutations tested.

However, the cost of building larger personalized assays is currently a limitation. Many commercially available tumour-informed assays are limited to testing for 10-100 cancer mutations primarily due to practical limitations such as the number of oligonucleotides that need to be designed and purchased to enrich for cancer mutations in the test sample. An ideal personalized assay should be made quickly (e.g., within a week, so as to provide actionable information for the patient), inexpensively (e.g., less than $1,000 for all materials), and to target as many somatic variants as possible (e.g., more than 1,000 variants, up to all somatic variants that may be identified from a cancer sample, such as 10,000 variants or 100,000 variants). Generating large numbers of oligonucleotides in parallel, such as on arrays, silicon wafers, or using semiconductor chips, can significantly reduce the cost of purchasing individual oligonucleotides through economies of scale. However, these methods require the production of many more oligonucleotides (typically, hundreds of thousands to millions) than is needed for a tumour informed assay (typically, one thousand to a hundred thousand).

Accordingly, the present disclosure results, in one example, from the realization that the problem of incorporating many (e.g., hundreds to tens of thousands) cancer mutations in a tumour informed assay in a cost-effective manner is solved by producing a large pool of oligonucleotides (e.g., using arrays, silicon wafers, semiconductor chips, or similar methods) that may be used for many patients. The pool may include oligonucleotides targeting a plurality of genomic regions from a plurality of different patients. Each oligonucleotide in the pool may contain target-specific sequence and patient-specific sequence. The patient-specific sequence may then be used to test only for those regions that are of interest in a specific patient, e.g. by use of oligonucleotides containing patient-specific and target-specific sequence to enrich or select for cancer mutations from that patient. In this way, the high initial cost of producing the pool may then be distributed across a plurality of patient samples, resulting in a fast, sensitive, and cost-effective tumor-informed assay.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

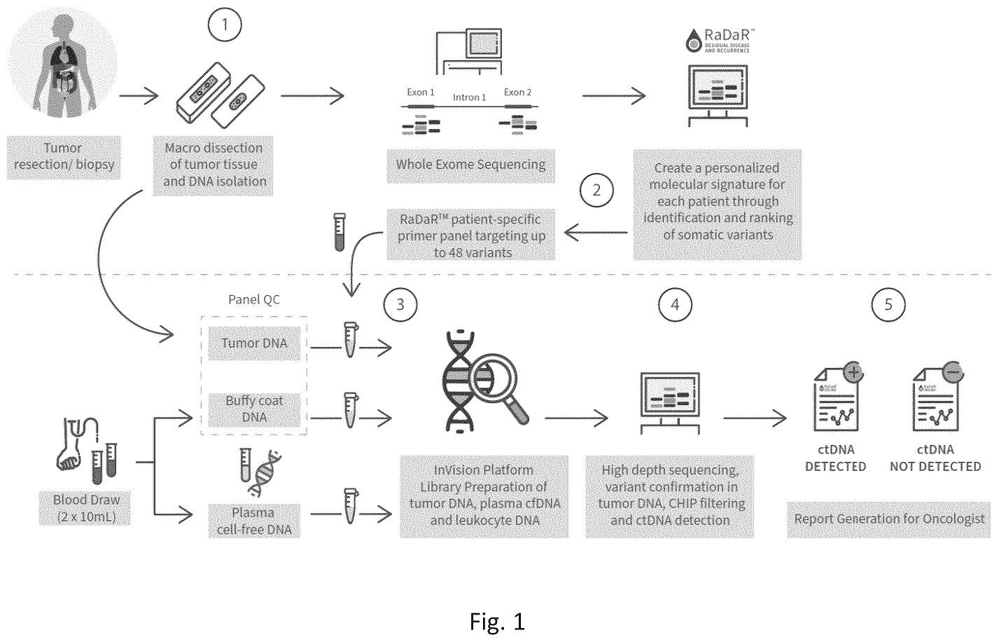

FIG. 1 depicts an example of a method of creating a tumour-informed assay that can benefit from methods as described herein. As shown in FIG. 1, a patient with cancer may have a tumour resection or biopsy, from which cancer DNA is isolated (1). This cancer DNA may be subjected to sequencing, such as whole genome sequencing, whole exome sequencing, or targeted sequencing, to generate sequence reads corresponding to the cancer DNA. Somatic variants (i.e., genetic variants unique to the cancer) may then be identified from the sequence reads to create a molecular signature for the cancer that may be used for tracking purposes. In this example, 48 variants are selected for inclusion in a primer panel (2). The primer panel may then be used to amplify those variants (or regions corresponding to those variants) in a patient blood or plasma sample (3). The amplified sample may then be sequenced, and the resulting sequence reads analyzed to determine whether those genetic variants are present in the blood or plasma sample (4) More detail can be found at least in International Patent Publications WO2022-029688A1 and WO2023-012521A1, the contents of which are hereby incorporated by reference in their entirety.

As shown in FIG. 1, the panel includes primers that will amplify 48 regions that may contain cancer-specific genetic variants. While increasing the number of regions to amplify will likely result in an increase in sensitivity (as it allows for detection of additional cancer-specific variants), another factor to consider is cost. Typically, due to economies of scale, there is a discount applied as one orders or produces larger and larger numbers of oligonucleotides. For example, some technologies may produce 2-100 oligonucleotides (i.e., 1-50 pairs of primers) for a cost of only $400, resulting in a panel that is cost-effective for the patient. 101-500 oligonucleotides may cost $800, 501-1000 oligonucleotides may cost $1200, and 6001-12,000 oligonucleotides may cost $3120. While selecting additional variants for inclusion in a panel may result in an increase in sensitivity, the additional cost may result in the panel not being competitive from a price standpoint. Unfortunately, as each panel is personalized to the patient, it is not currently possible using conventional techniques to produce a large number of specific oligonucleotides beforehand to realize any oligonucleotide cost savings from economies of scale.

However, the inventors have recognized and appreciated that a selective sequencing technique may be employed to enable the use of large numbers (e.g., 1,000, 5,000, 10,000, 100,000, 1,000,000, 10,000,000) of oligonucleotides in a targeting reaction (e.g. primer pairs in an amplification reaction), thereby allowing the cost-effective production of a large quantity of oligonucleotides; but, only those oligonucleotides that are specific to somatic genetic variants from a specific patient's cancer sample may enable that specific target DNA to be enriched, amplified, and sequenced. Such oligonucleotides may be made at large scale cost effectively in many ways for example using silicon-based DNA synthesis or inkjet-based oligonucleotide synthesis on arrays. In this way, oligonucleotides corresponding to large swathes of the genome can be produced or purchased beforehand, allowing one to spread the initially high cost of the oligonucleotides over many patients instead of a single patient, simultaneously increasing the number of genetic variants that may be included in a panel while significantly reducing the cost.

Accordingly, the present disclosure provides methods for selective sequencing of a plurality of target regions from a first patient, each of the target regions containing, or suspected of containing, one or more cancer specific variants. In some embodiments, these cancer specific variants are variants previously detected within a cancer sample from the patient. The cancer specific variant may be a SNV, an indel, or a fusion variant. Such cancer specific variants may be identified by sequencing a test sample from a first patient of: (i) DNA or RNA isolated from a tissue biopsy that comprises cancer cells, (ii) DNA or RNA isolated from a cancer tissue obtained at surgery that comprises cancer cells, (iii) sequencing cell-free DNA or RNA, (iv) DNA or RNA isolated from other suitable fluid samples such as a blood or bone marrow aspirate from an individual with hematological cancer, or (v) DNA or RNA isolated from circulating cancer cells, wherein the sample is from the same patient, e.g., prior to any treatment. The variants may be identified by whole genome sequencing of tumor tissue or by sequencing a smaller genome region e.g. an exome. A control sample of non-cancerous DNA or RNA is sequenced, for example buccal swab DNA, whole blood DNA, adjacent non-cancerous DNA, i.e. from tissue that is adjacent to a tumor that appears normal, and compared to the test sample. The sequencing of these control samples may be performed at the same time as the test sample or it may be performed before or after sequencing the test sample. Sequence variants that are detected in the test samples (cancer DNA) and not the control samples (non-cancerous DNA) may be selected as candidate variants as they are likely to be tumor specific. Variants that are detected in the control samples (non-cancerous DNA) may be excluded as they are likely to not be cancer specific.

In many embodiments a variant or sequence variation is a variant that is present at a frequency of less than 50%, relative to other molecules in the sample. Many sequence variations, e.g., indels and nucleotide substitutions, are substantially identical to the molecules that do not contain the sequence variation. In some cases, a particular sequence variation may be present in a sample at a frequency of less than 20%, less than 10%, less than 5%, less than 1%, less than 0.5%, less than 0.1%, less than 0.05% or less than 0.01%.

The term “target region” refers to a region of DNA that contains or is suspected of containing one or more sequence variations, but excluding “control regions”. A target region may be less than 500 base pairs in length. Preferably, a target region may be less than 160 base pairs in length, or 50-160 bp, corresponding to the fragment size of cell-free DNA. Sequencing a plurality of target regions from a first patient, each of the target regions containing, or suspected of containing, one or more cancer specific variants is desirable because analysis of multiple variants helps increase the sensitivity and specificity of tests for cancer DNA. For example, by reducing the chance of a false positive.

Generally, the methods comprise a step of providing a pool of oligonucleotides. The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example. The term “pool”, as used herein, refers to the combination or mixture of two or more species or sub-populations of oligonucleotide such that the molecules within those species are interspersed with one another in solution.

In some embodiments, the pool of oligonucleotides comprises a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient.

In some embodiments the pool of oligonucleotides may also comprise a second (or third or fourth etc.) sub-population of oligonucleotides having the same features as the first sub-population, but targeting a different patient or patients, or a second tumor from the first patient. For example, a second sub-population of oligonucleotides that target the plurality of target regions from a second patient, wherein each member of the second sub-population of oligonucleotides comprises a first sequence that is complementary to one of the plurality of target regions from the second patient and an identifier sequence specific to the second patient, and so on.

An oligonucleotide that “targets” one or a plurality of target regions may hybridize to the target region to which it is complementary. If two nucleic acids are “complementary,” they hybridize with one another under high stringency conditions. In many cases, two sequences that are complementary have at least 10, e.g., at least 12 or 15 nucleotides of complementarity. As used herein, the term “hybridizing” or “hybridization” refers to any process by which a strand of nucleic acid binds with a complementary strand through base pairing. For example, “hybridization” refers to a process in which a region of nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strand regions in a hybridization reaction.

The term “identifier sequence” as used herein refers to a sequence of the oligonucleotide which is specific to a particular sub-population of oligonucleotides and may act as a tag or barcode to identify said sub-population of oligonucleotides. For example, a patient-specific sequence may be an identifier sequence. For example, the identifier sequence may be specific to a particular sub-population of oligonucleotides which is specific to a first patient and may therefore act as a tag or barcode to identify said patient. Therefore “identifier sequence specific to the first patient” as used herein can equally mean “identifier sequence specific to a first sub-population of oligonucleotides”. An identifier sequence which is specific to the first patient will be present in a sub-population of oligonucleotides comprising a sequence complementary to a target region from the same patient. For example, in a first sub-population of oligonucleotides that target the plurality of target regions from the first patient, each member of the first sub-population of oligonucleotides may comprises a first sequence that is complementary to one of the plurality of target regions from the first patient and a 5′ tail that comprises an identifier sequence specific to the first patient, such that after an amplification step the sequence of the 5′ tail is present in the amplicons. Identifier sequences can also be ligated on to the products. Therefore once the oligonucleotides have hybridized to the target region, the identifier sequence will also be present in the resulting duplex, allowing identification of the patient of origin. If identifier sequences are present, the products derived from different patients can be pooled prior to sequencing. This applies mutatis mutandis to the second or subsequent sub-population of oligonucleotides.

Generally the methods further comprise contacting a test sample, or amplified test sample, from the first patient with the pool of oligonucleotides. In preferred embodiments, the test sample comprises cell free DNA. Contacting the sample may comprise mixing the test sample and the pool of oligonucleotides, or may comprise adding the test sample to the pool of oligonucleotides, or vice versa. The oligonucleotides in the first sub-population of oligonucleotides (which comprise comprises sequences that are complementary to one of the plurality of target regions from the first patient) will hybridize to the target regions within the test sample.

In some embodiments, the test samples are then amplified to produce an amplified test sample. The amplified test sample will then include the identifier sequences in the resulting duplex, allowing identification of the patient of origin. The term “amplifying” as used herein refers to generating one or more copies of a target nucleic acid, using the target nucleic acid as a template. The test samples may be amplified by PCR or any amplification technique known in the art.

In general, the methods then comprise contacting the test sample (or amplified test sample) with oligonucleotides each comprising a sequence that is complementary to the identifier sequence specific to the first patient. These oligonucleotides hybridize to the test sample or amplified test sample at the complementary region. For example, the oligonucleotides may be a PCR product derived from the test sample, or to another oligonucleotide which is itself hybridized to the test sample. In some embodiments, the oligonucleotides may comprise additional sequencing adaptors that provides compatibility with a particular sequencing platform.

The methods generally comprise selectively sequencing the plurality of target regions from the first patient, optionally thereby identifying the presence or absence of the cancer specific variants. The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained. The sequencing step may be done using any convenient sequencing method, preferably a next generation sequencing method. In some embodiments the next generation sequencing method may result in at least 100,000, at least 500,000, at least 1M at least 10M at least 100M, at least 1B or at least 10B sequence reads per reaction. In some cases, the reads may be paired-end reads. The term “next generation sequencing” refers to the so-called highly parallelized methods of performing nucleic acid sequencing and comprises the sequencing-by-synthesis, sequencing by binding, or sequencing-by-ligation platforms currently employed by Illumina, Life Technologies, Pacific Biosciences and Roche, etc. Next generation sequencing methods may also include, but not be limited to, nanopore sequencing methods such as offered by Oxford Nanopore or electronic detection-based methods such as the Ion Torrent technology commercialized by Life Technologies.

The term “sequence read” refers to the output of a sequencer. A sequence read typically contains a string of Gs, As, Ts and Cs, of 50-1000 or more bases in length and, in many cases, each base of a sequence read may be associated with a score indicating the quality of the base call.

“Selectively” sequence means preferentially sequencing nucleic acid molecules from or derived from the first patient's target regions, i.e. the test patient. Sequencing of nucleic acid molecules from or derived from the other patients is avoided by using patient-specific oligonucleotides that are specific for the first patient, thereby enriching the sample to be sequenced for the regions targeted by those oligonucleotides. The sequence of the cancer specific variant may then be determined during the selective sequencing, as well as determining its presence or absence, e.g., in a sequence read.

Therefore, the assay remains personalized (because a personalized set of target regions is targeted for each patient) but the costly step of generating a personalized oligonucleotide panel for each patient is avoided. Instead, a single pool of oligonucleotides (containing a plurality of sub-populations) can be used to selectively sequence only nucleic acids from a single sub population by using patient-specific oligonucleotides that are specific for the that patient's sub populations. In some embodiments, the method further comprises performing the same method for a second, different, patient using a test sample from the second patient and patient-specific oligonucleotides specific to the second patient.

In such embodiments, the methods further comprise contacting a test sample from the second patient with the pool of oligonucleotides; contacting the test sample with patient-specific oligonucleotides each comprising a sequence that is complementary to the patient-specific sequence specific to the second patient; and using the patient-specific oligonucleotides, and selectively sequencing the plurality of target regions from the second patient, optionally thereby identifying the presence or absence of the cancer-specific variants. All embodiments described above in relation to the first patient and associated specific oligonucleotides may apply mutatis mutandis to the second patient and associated specific oligonucleotides.

As the skilled person would understand, many techniques for amplifying, processing, and next generation sequencing of nucleic acids are available. These techniques can be utilized in the methods described herein. Now the overarching concept has been described, the methods will be further explained in relation to specific techniques of carrying them out.

Embodiments of the disclosure may comprise tumour-informed assays that use oligonucleotides that target somatic variations previously identified in a patient's cancer. Accordingly, in a sixth aspect, provided herein is a method of designing a plurality of oligonucleotides for use with selective sequencing, the method comprising the steps of:
a) sequencing, or having sequenced, cancer DNA from a plurality of samples;
b) calling a plurality of somatic variants from the sequenced cancer DNA to generate a plurality of genetic variations associated with the cancer DNA from the plurality of samples;
c) designing a plurality of oligonucleotides corresponding to the plurality of somatic variations or a subset of the plurality of genetic variations, wherein each oligonucleotide contains a target-specific region corresponding to one of the identified somatic variants, and at least one identifier sequence specific to a patient; and
d) generating or having generated a pool of the designed oligonucleotides.

Methods of designing a plurality of oligonucleotides may also be combined with the methods of selective sequencing provided in the other aspects. In some embodiments of any of the aspects, the method can comprise designing a plurality of oligonucleotides for use with selective sequencing.

The method of any aspect may further comprise an initial step of designing the sub-populations of oligonucleotides, the initial step comprising:
a) sequencing cancer DNA from a plurality of samples;
b) calling a plurality of somatic variants from the sequenced cancer DNA to generate a plurality of genetic variations associated with the cancer DNA from the plurality of samples;
c) designing a plurality of oligonucleotides corresponding to the plurality of somatic variations or a subset of the plurality of genetic variations, wherein each oligonucleotide contains a target-specific region corresponding to one of the identified somatic variants, and at least one identifier sequence specific to a patient; and
d) generating or having generated a pool of the designed oligonucleotides.

The plurality of oligonucleotides designed by these methods may be the pool of oligonucleotides, or any sub-population of oligonucleotides referred to in any of the aspects herein.

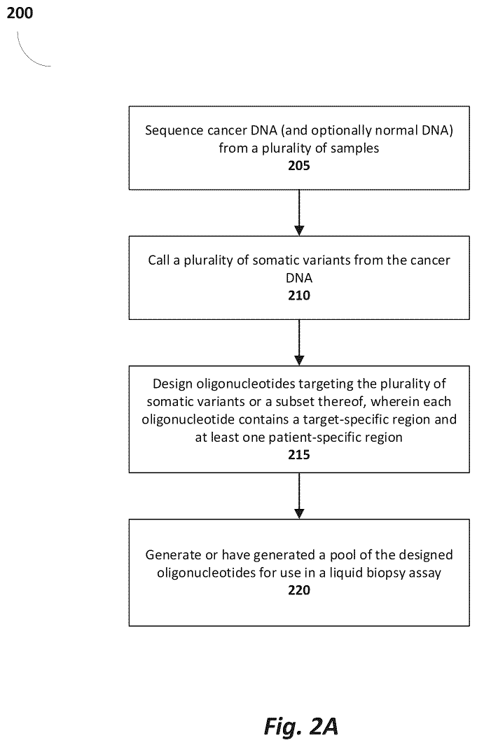

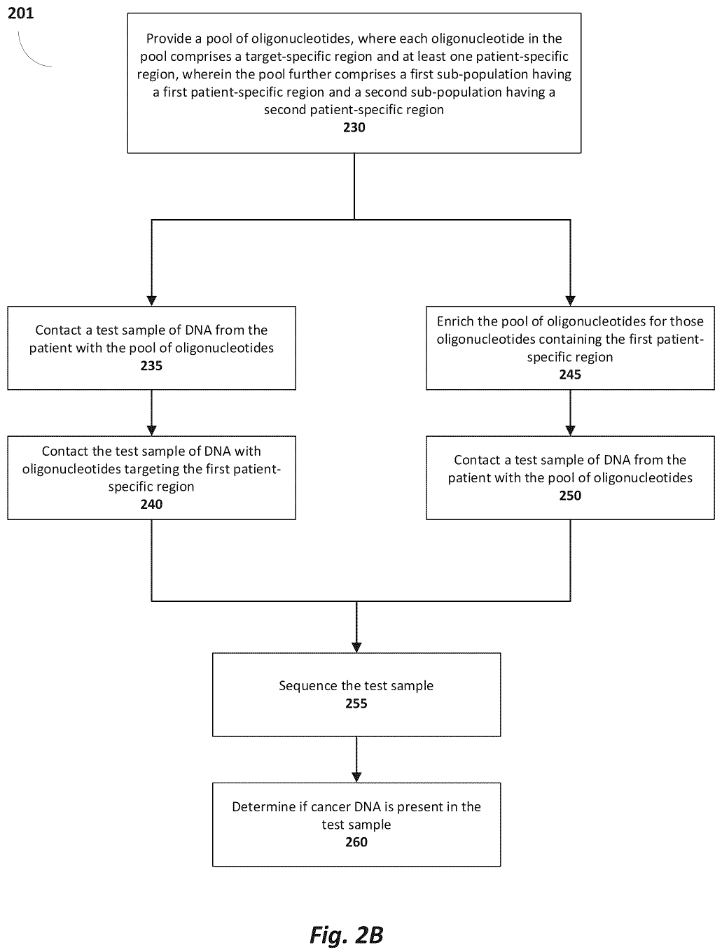

FIG. 2A depicts an example embodiment of a method 200 of generating a pool of oligonucleotides according to the disclosure. As shown in FIG. 2A, the method 200 can begin by sequencing cancer DNA from a plurality of samples, such as tumor samples from cancer patients (step 205). Somatic variants may then be called from the sequenced cancer DNA to generate a plurality of genetic variations associated with the cancer DNA from the plurality of samples (step 210). These somatic variants thus represent different somatic variants from a variety of different cancers and cancer types. Optionally, the somatic variants may be ranked or filtered, and a subset may be selected, such as described in International Patent Publication No. WO2023-012521. The method 200 may then continue by designing a plurality of oligonucleotides corresponding to the plurality of somatic variations or a subset of the plurality of somatic variations (step 215). Each oligonucleotide contains a target-specific region corresponding to one of the identified somatic variants, and at least one identifier or patient-specific region that can uniquely identify a subset of the plurality of oligonucleotides. The method 200 can then continue by generating a pool of the designed oligonucleotides (step 220), or having a pool of the designed oligonucleotides generated. In some embodiments, the oligonucleotides may be generated by a method that creates all of the oligonucleotides in parallel, including but not limited to array-based approaches, silicon wafers, or using semiconductor chips.

Sequencing of cancer DNA from a plurality of samples (step 205) can be performed in a variety of ways. In some embodiments, a plurality of tumor biopsy samples from a plurality of cancer patients can be collected and subjected to (e.g.) whole genome sequencing or whole exome sequencing. As previously described herein, sequencing may be performed using “next generation sequencing”, which refers to the so-called highly parallelized methods of performing nucleic acid sequencing and can comprise sequencing-by-synthesis, sequencing by binding, or sequencing-by-ligation platforms as further described herein.

Somatic variation calling (step 210) is a bioinformatics process used to identify genetic differences in a tumor sample that are unique to the tumor. “Calling” a plurality of somatic variants from the sequenced cancer DNA may involve identifying and selecting somatic variants from the sequenced cancer DNA. In some embodiments, somatic variation calling comprises identifying genetic differences between a tumor sample and a matched normal sample. To perform somatic variation calling, sequencing data from the tumor and optionally, normal samples is obtained. The data may be preprocessed to remove any artifacts or low-quality reads. Next, the reads are aligned to a reference genome using specialized alignment tools. After alignment, the aligned data is analyzed to identify somatic mutations, including but not limited to single nucleotide variants (SNVs), insertions, deletions, and structural variants. Various software tools may be used to perform somatic variant calling, including but not limited to Mutect2, Varscan, and Strelka. Post-calling filtering and quality control steps may also be applied to reduce false positives and ensure the reliability of the identified somatic variations.

Optionally, the called somatic variants identified from the cancer sample (or target regions containing those somatic variants) may be ranked or filtered, and a subset may be selected for use with designing oligonucleotides. In some embodiments, the somatic variations or target regions may be filtered, scored, or ranked based on one or more of clonality, allele fraction within the cancer sample, mappability, estimated background error rate wherein sequence variations that show evidence of sequence or PCR polymerase error rate are penalized or filtered, estimated rate of high signal background events wherein variations that show DNA damage or early-cycle PCR errors are penalized or filtered, distance from another selected variant, avoidance of regions known to not be optimal, predictive ability to sequence, presence in a region of known copy number gain or amplification, proximity of any germ line (not somatic) variants, likelihood of being somatic, and other criteria, as described in more detail in International Patent Publication WO2023-012521, the contents of which are hereby incorporated by reference in their entirety.

In some embodiments, designing oligonucleotides targeting each somatic variant (step 215) comprises designing nucleotide sequences targeting each somatic variant. Each oligonucleotide may include a target-specific portion that includes sequence complementary to a target region containing a somatic variant, as the target-specific portion is intended to bind to, capture, or otherwise enrich for DNA containing that somatic variant, as further described herein. Each oligonucleotide may also include a patient-specific region that includes sequence that is not specific to any target region but may be used to uniquely identify or enrich for DNA containing that patient-specific region. or uniquely identify or enrich the target specific oligonucleotides for the patient. Accordingly, in some embodiments, the patient-specific region is an identifier sequence that may be used to match populations of oligonucleotides targeting certain regions with particular patients that may also have somatic variants in those regions. The oligonucleotides may contain at least two different patient-specific sequences, thus creating a first sub-population of oligonucleotides having a first patient-specific sequence and a second sub-population of oligonucleotides having a second patient-specific sequence.

In some embodiments, generating a pool of the designed oligonucleotides (step 220) comprises using a method that creates all of the oligonucleotides in parallel, including but not limited to array-based approaches, silicon wafers, or using semiconductor chips. The designed oligonucleotides may also be generated by a third party that provides such a generation service. These approaches leverage the high-density arrangement of microfluidic wells or specific chemical reactions on a solid surface to synthesize thousands or even millions of oligonucleotides simultaneously. In array-based methods, such as DNA microarrays, specific DNA sequences are synthesized on a solid surface in a spatially addressable manner. Silicon wafers and semiconductor chips use microfabrication techniques to create an array of microreactors, where each microreactor can independently generate an oligonucleotide. Such parallel synthesis strategies enable rapid and cost-effective production of many diverse oligonucleotides, but the resulting oligonucleotides are in a single pool and thus may still not be cost effective to be used with a single patient. Accordingly, methods according to the disclosure employ selective sequencing techniques to allow usage of the pool for a variety of patients but enriching only for targets of interest for a given patient.

FIG. 2B depicts an example embodiment of a method 201 of selectively sequencing a plurality of target regions from a first patient. The method 201 can begin by providing a pool of oligonucleotides, where each oligonucleotide in the pool comprises a target-specific region and at least one patient-specific region, wherein the pool further comprises a first sub-population having a first patient-specific region (an identifier sequence specific to the first patient) and a second sub-population having a second patient-specific region (an identifier sequence specific to the second patient) (step 230). In some embodiments, the pool of oligonucleotides targets a plurality of cancer-specific variants identified from a plurality of patients (including the first patient), wherein there may be different variants for each patient. The pool of oligonucleotides may be generated according to the method 200 of FIG. 2A, for example.

In some embodiments, the method 201 can continue by contacting a test sample of DNA from a patient with the pool of oligonucleotides (step 235). In these embodiments, all of the oligonucleotides in the pool (including multiple sub-populations of patient-specific sequence) may bind to DNA in the test sample via the target-specific sequence in each oligonucleotide. In these embodiments, the test sample may then be contacted (step 240) with oligonucleotides targeting only a first sub-population of oligonucleotides containing a patient-specific sequence. These oligonucleotides may comprise PCR primers, for example, that selectively bind to and amplify those oligonucleotides bound to certain target regions that also contain the patient-specific sequence, thus enriching the sample for those target regions. Of course, contacting the test sample of DNA with either the patient-specific oligonucleotide (step 240) or the pool of oligonucleotides (step 235) could be done in either order.

In other embodiments, the method 201 can continue by instead enriching the pool of oligonucleotides for a first sub-population containing a single patient-specific sequence (step 245), as further described herein. In these embodiments, those oligonucleotides containing a certain patient-specific sequence are first selected for, e.g., through amplification, hybridization, making those oligonucleotides functional (e.g., by combining with biotin) or another technique, as further described herein. In these embodiments, the method 201 can continue by contacting a test sample of DNA from the patient with the modified pool of oligonucleotides, thus enriching the test sample for targets corresponding to oligonucleotides having that patient-specific sequence. Provided therein is therefore a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. enriching the pool of oligonucleotides for those oligonucleotides containing the identifier sequence specific to the first patient;
c. contacting a test sample from the first patient with the enriched pool of oligonucleotides;
d. selectively sequencing the plurality of target regions from the first patient, optionally thereby identifying the presence or absence of the cancer specific variants.

In any embodiment, the test sample of DNA may then be sequenced (step 255). The sequence reads may then be analyzed to determine whether cancer DNA is present in the test sample (step 260), e.g., by calling one or more variants as present in the test sample, or combining evidence across all of the variants tested, as further described herein and in International Patent Publication WO2023-012521.

In any embodiment, the methods herein may be used to selectively sequence a plurality of target regions from 2 to 1000 patients, or from 5 to 500 patients, or from 5 to 100 patients. In any embodiments, the pool of oligonucleotides may comprise oligonucleotides targeting a plurality of genomic regions from 2 to 1000 patients, or from 5 to 500 patients, or from 5 to 100 patients.

In any embodiment, “one or more cancer specific variants” may refer to from 10 to 1,000,000 cancer specific variants, or from 100 to 100,000 cancer specific variants, or from 500 to 50,000 cancer specific variants, or from 1,000 to 10,000 cancer specific variants, or from 1,000 to 20,000 cancer specific variants.

### Example Embodiments of Methods for Selective Sequencing of a Plurality of Target Regions as Described Herein, Using PCR Techniques

In some aspects, PCR techniques can be used in the general methods described herein. The features and definitions already provided for the general method apply equally to these aspects.

A “polymerase chain reaction” or “PCR” is an enzymatic reaction in which a specific template DNA is amplified using one or more pairs of sequence specific primers. A “multiplex polymerase chain reaction” or “multiplex PCR” is an enzymatic reaction that employs two or more primer pairs for different targets templates. If the target templates are present in the reaction, a multiplex polymerase chain reaction results in two or more amplified DNA products that are co-amplified in a single reaction using a corresponding number of sequence-specific primer pairs.

In such embodiments the pool of oligonucleotides comprises “primer oligonucleotides” or “primers”. “Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers are extended by a DNA polymerase. Primers are generally of a length compatible with their use in synthesis of primer extension products, and are usually in the range of 8 to 200 nucleotides in length, such as 10 to 100 or 15 to 80 nucleotides in length. A primer may contain a 5′ tail that does not hybridize to the template.

Primers are usually single-stranded for maximum efficiency in amplification, but may alternatively be double-stranded or partially double-stranded. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

In some aspects is therefore provided methods for selective sequencing of a plurality of target regions from a first patient, each of the target regions containing, or suspected of containing, one or more cancer specific variants, the method comprising providing a pool of oligonucleotides, wherein the pool of oligonucleotides comprises primer oligonucleotides.

In some embodiments the pool of oligonucleotides comprises a first sub-population of primer oligonucleotides. In some embodiments this includes pairs of forward and reverse primers, or a single forward primer or a single reverse primer, each targeting the plurality of target regions. In other embodiments this may include a pair of primers comprising one target specific primer and one common primer that is not target specific. Each primer comprises a first sequence that is complementary to one of the target regions from the first patient and an identifier sequence specific to the first patient. An identifier sequence which is specific to the first patient will be present in the sub-population of primers comprising a sequence complementary to a target region from the same patient. For example, in a first sub-population of primer oligonucleotides that target the plurality of target regions from the first patient, each primer may comprise a first sequence that is complementary to one of the plurality of target regions from the first patient and a 5′ tail that comprises an identifier sequence specific to the first patient, such that PCR the sequence of the 5′ tail is present in the PCR product. Identifier sequences can also be ligated on to the products. Therefore, after PCR has been conducted the identifier sequence will be present in PCR product, allowing identification of the patient of origin. If identifier sequences are present, the PCR products derived from different patients can be pooled prior to sequencing. This applies mutatis mutandis to the second or subsequent sub-population of oligonucleotides. In some embodiments, the identifier sequence specific to the first patient is present in both the forward and reverse primer. In some embodiments, the identifier sequence specific to the first patient is present in only the forward primer.

In some embodiments the pool of oligonucleotides may also comprise a second (or third or fourth etc.) sub-population of primers having the same features as the first sub-population, but targeting a different patient or patients. For example, in a second sub-population of primers, each primer comprises a first sequence that is complementary to one of a plurality of target regions from a second patient and an identifier sequence specific to the second patient, and so on.

In some embodiments, the methods comprise contacting a test sample from the first patient with the pool of oligonucleotides. The test sample may be any of those described herein. In some embodiments, this comprise adding the primer oligonucleotides to the test sample such that they hybridize to their target regions.

In some embodiments, the methods comprise performing PCR (a first round of PCR) on the test sample comprising the pool of oligonucleotides to produce an amplified test sample, also known as amplicons, or PCR products. The skilled person is able to determine suitable PCR conditions. “PCR conditions” are the conditions in which PCR is performed, and include the presence of reagents (e.g., nucleotides, buffer, polymerase, etc.) as well as temperature cycling (e.g., through cycles of temperatures suitable for denaturation, renaturation and extension), as is known in the art. Any suitable PCR technique can be used, for example multiplex PCR, long-range PCR, single-cell PCR, fast-cycling PCR, methylation-specific PCR (MSP), hot start PCR, high-fidelity PCR, RAPD: Rapid amplified polymorphic DNA analysis, RACE: Rapid amplification of cDNA ends, in situ PCR, differential display PCR, real-Time PCR (quantitative PCR or qPCR), reverse-transcriptase (RT-PCR), nested PCR, assembly PCR, or asymmetric PCR.

In some embodiments, the method further comprises enriching the amplified test sample for amplicons derived from the first patient's target regions by contacting the amplified test sample with patient-specific primer pairs and performing PCR, wherein one or both primers comprises a sequence that is complementary to a identifier sequence specific to the first patient present in the first sub-population of oligonucleotides from the first round of PCR. This second round of PCR uses primers that target the identifier sequence specific to the first patient that was introduced during the first round of PCR. Therefore only amplicons derive from the first patient's target regions of interest will be amplified by the second round of PCR. In some embodiments, the patient-specific primers further comprise sequencing adaptors. The sequencing adaptors provide compatibility with a particular sequencing platform, optionally an Illumina sequencing platform, a PacBio Onso sequencing platform, an Element sequencing platform or an Ultima sequencing platform, among others.

A patient-specific primer used in a preferred embodiment of the invention may therefore comprise a sequence that is complementary to a identifier sequence specific to the first patient present in the first sub-population of oligonucleotides from the first round of PCR and a sequencing adaptor.

In some embodiments the methods comprise selectively sequencing the enriched sample to determine the sequence of the plurality of target regions from the first patient, optionally thereby identifying the presence or absence of the cancer specific variants. “Selectively” sequence means preferentially sequencing nucleic acid molecules from or derived from the first patients target regions, i.e. the test patient. Sequencing of nucleic acid molecules from or derived from the other patients regions is avoided by using patient-specific primers that are specific for the first patients regions. The sequence of the cancer specific variant is determined during the selective sequencing, as well as determining its presence or absence.

In some embodiments, the method further comprises performing the same PCR based method for a second, different, patient using test sample from the second patient and patient-specific primers specific to the second patient.

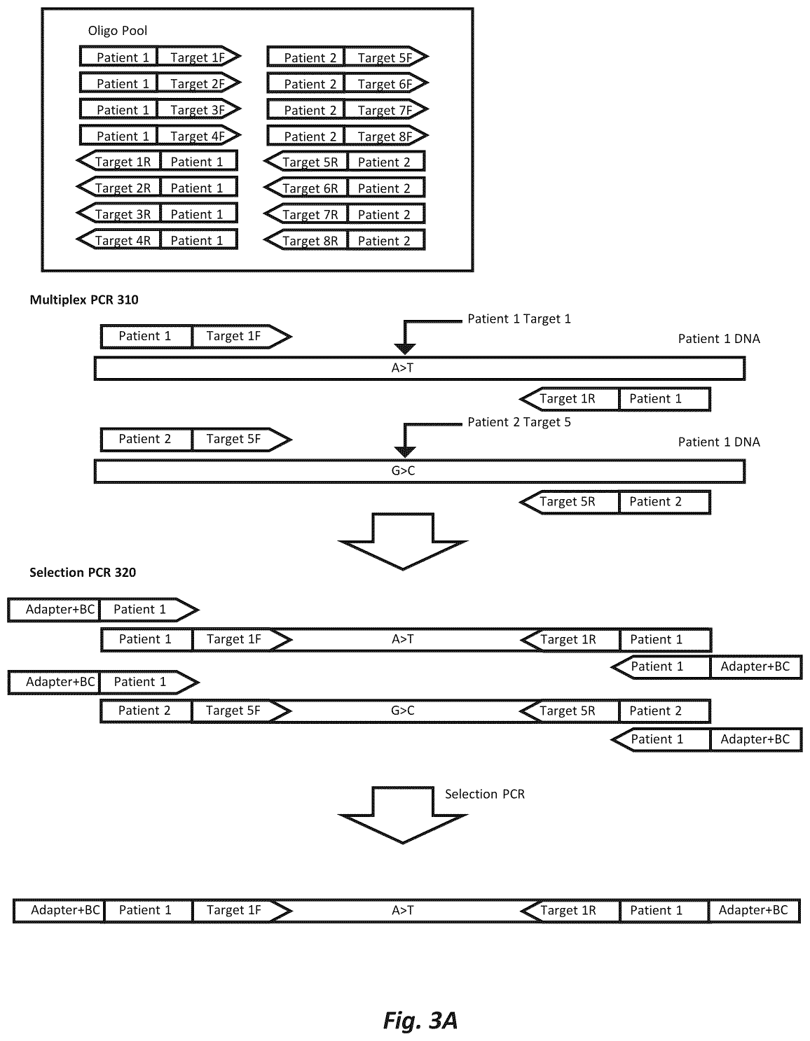

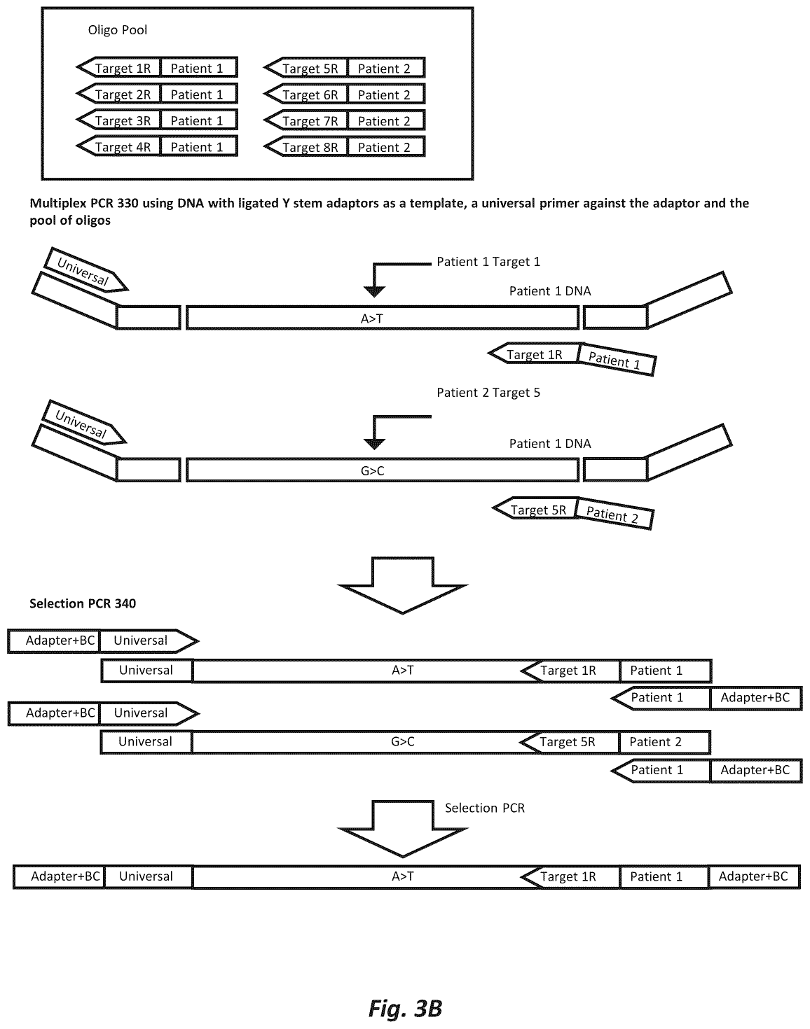

FIG. 3A shows an example embodiment of a selective sequencing method as described herein. In this embodiment, the oligonucleotides in the pool are PCR primers and they include a patient-specific identifier (e.g. “Patient 1”) and a target-specific portion (e.g. “Target 1”). Four forward primers (F) for patient 1 targets, four reverse primers for patient 1 targets, four forward primers for patient 2 targets and four reverse primers for patient 2 targets are shown in the “oligo pool”, although a practical oligo pool would include significantly more primers, as further described herein. The pool of primers is then combined with a test sample of DNA from patient 1 in a multiplex PCR reaction 310. As shown in FIG. 3A, only the primers for “Patient 1 Target 1” and “Patient 2 Target 5” are shown in the multiplex PCR reaction, but all primers in the oligo pool would be present in the multiplex PCR because the pool is contacted with the test sample of DNA. In this example, the primers bind to DNA containing target 1 and target 5, and so both target 1 and target 5 are amplified from the DNA of patient 1 in a the multiplex PCR reaction 310 to produce an amplified test sample.

The amplified test sample may then be contacted with primer pairs specific to the patient specific sequences on the 5′ ends of the oligos targeting the regions of interest for patient 1 and further “selective” PCR 320 is performed. Through this additional selective PCR, target region 1 is amplified (along with other patient 1 targets targeted by the oligo pool) and sequencer adaptors and patient specific barcodes (“BC”) are added whilst region 5 (and all regions for other patients or patient identifiers within the oligo pool) is not amplified nor do they have sequencer adapters or barcodes attached. Thus, the selective PCR 320 has enriched the test sample only for DNA containing the targets from patient 1.

FIG. 3B shows another example embodiment of a selective sequencing method as described herein wherein the oligonucleotide pool may contain only a single forward or reverse primer for a given target site. In this embodiment, the oligonucleotides in the pool are PCR primers and they include a patient-specific identifier (e.g. “Patient 1”) and a target-specific portion (e.g. “Target 1”). Four reverse primers for patient 1 targets and four reverse primers for patient 2 targets are shown in the “oligo pool”, although a practical oligo pool would include significantly more primers, as further described herein. The pool of primers are combined with a test sample of DNA from patient 1 that has previously had adaptors ligated (such as Y-stem adaptors), wherein the adaptors further include a universal primer sequence, along with a primer targeting the universal sequence primer within the adaptor in a multiplex PCR reaction 330. Just the primers for “Patient 1 Target 1” and “Patient 2 Target 5” are shown in the multiplex PCR reaction 330, but all primers in the pool would be present in the multiplex PCR. In this example, the universal primer binds all ligated adaptors and the primers from the oligo pool bind their respective targets and both target 1 and target 5 are amplified from the DNA of patient 1 producing an amplified test sample without the need to have both forward and reverse primers present for each target in the oligo pool.

The amplified test sample may then be contacted with primer pairs, wherein one member of the pair is specific to the patient specific sequence on the 5′ end of the oligos targeting the regions of interest for patient 1 and the other member of the pair targets the universal primer sequence. Amplifying this test sample performs a selection PCR 340. Through this selection PCR 340, target region 1 is amplified (along with other patient 1 targets) and sequencer adaptors and patient specific barcodes (“BC”) are added whilst region 5 (and all regions for other patients within the oligo pool) is not amplified nor do they have sequencer adapters or barcodes attached. Thus, the selection PCR 340 has enriched the test sample only for DNA containing the targets from patient 1.

In some embodiments, the oligonucleotides may be amplified by rolling circle amplification instead of by PCR. In such an embodiment, rolling circle amplification is used to selectively amplify the oligonucleotides targeted to a patient specific sequence from one patient. These oligonucleotides can then be used for target capture, for example using biotin. In such an embodiment, single stranded oligonucleotides would first be circularized. This may be achieved using a split adapter to bring the two ends of the molecule together facilitating ligation. In one embodiment all oligonucleotides would be circularized using a common splint complementary to both ends of the oligonucleotides. In an alternative embodiment, the ends of each oligonucleotide would contain the patient specific sequences and a patient specific splint would be used resulting in just oligonucleotides designed against targets for one patient being circularized and ultimately amplified. In some embodiments the primers used to initiate rolling circle amplification would be targeted to a patient specific sequence. Rolling circle amplification may be performed with biotinylated nucleotides and following amplification, the oligonucleotides may be digested into shorter molecules to enable their use for target capture.

### Methods for Selective Sequencing of a Plurality of Target Regions as Described Herein Using DNA Hybridization Capture Techniques

In some aspects, hybridization capture techniques can be used in the general methods described herein. The features and definitions already provided for the general method apply equally to these aspects.

“Hybridization capture”, “hybrid capture” or “target enrichment” is a next generation sequencing method that uses oligonucleotide baits or probes, such as biotinylated baits, to hybridize to target regions of interest through complementary base pairing. Once the target region of interest has been captured by the bait (e.g. the bait oligonucleotide has hybridized to the target region) the bait can be separated from the rest of the sample to create an enriched sample. Different methods of separation are known, for example via magnetization with a streptavidin-biotin binding complex, or in solid phase where the baits are bound to a solid support such as a glass slide.

In some aspects is provided methods for selective sequencing of a plurality of target regions from a first patient, each of the target regions containing, or suspected of containing, one or more cancer specific variants, the method comprising providing a pool of oligonucleotides, wherein the pool of oligonucleotides comprises oligonucleotide baits, also known as oligonucleotide probes. The oligonucleotide baits are oligonucleotides that can be used to retrieve specific target sequences, for sequencing for example. The baits are complementary for the target region such that the baits hybridize with the target region and not other regions. The baits may be single stranded DNA or single stranded RNA. The oligonucleotide baits may be 10 to 20, 21 to 30, 31 to 40, 41 to 50, 51 to 60, 61 to 70, 71 to 80, 80 to 100, 100 to 150, 170, or 150 to 200 nucleotides in length, for example. In preferred embodiments, the oligonucleotide baits are 100 to 200 nucleotides in length. The oligonucleotide baits may further comprise a binding moiety, such as biotin.

In some embodiments, the pool of oligonucleotides comprises a first sub-population of bait oligonucleotides, wherein the first sub-population of oligonucleotides comprises oligonucleotide baits that target the plurality of target regions from the first patient, wherein each member of the first sub-population of oligonucleotides comprises a first sequence that is complementary to one of the target regions from the first patient and an identifier sequence specific to the first patient. In embodiments where the target region is in double stranded DNA, each bait will be complementary to one strand of target DNA, and will match the sequence of the opposite strand of target DNA. In some embodiments the bait oligonucleotides are biotinylated. Biotinylated means at least one biotin molecule is conjugated, for example covalently attached, to the bait oligonucleotide. Biotinylation can occur through chemical or enzymatic means.

In some embodiments the pool of oligonucleotides may also comprise a second (or third or fourth etc.) sub-population of oligonucleotides having the same features as the first sub-population, but targeting a different patient or patients. For example, a second sub-population of bait oligonucleotides, wherein the second sub-population of oligonucleotides comprises oligonucleotide baits wherein each member of the second sub-population of oligonucleotides comprises a first sequence that is complementary to one of a plurality of target regions from a second patient and an identifier sequence specific to the second patient.

In some embodiments, the methods further comprise a step of performing PCR on the test sample from the first patient at this point to produce an amplified test sample.

The methods may then comprise contacting a test sample from the first patient or an amplified test sample derived from the test sample with the pool of oligonucleotides. In preferred embodiments this involves adding the oligonucleotide baits to the test sample (if in liquid state) or adding the test sample to the oligonucleotide baits (if in solid state and the baits are immobilized).

The methods may then comprise contacting the test sample or amplified test sample with patient-specific oligonucleotides each comprising a binding moiety and a sequence that is complementary to the identifier sequence specific to the first patient, to produce a tagged test sample. In some embodiments, instead of the binding moiety (which is the means of separating the baits once they have captured their target) being part of the oligonucleotide baits, the binding moiety is comprised in the patient-specific oligonucleotides. This enables only the target regions from the first patient to be separated by use of the oligonucleotide baits. In preferred embodiments, the patient-specific oligonucleotides comprise a binding moiety such as biotin. In some embodiments the patient-specific oligonucleotides and the pool of oligonucleotides are combined and hybridized prior to being added to the test sample or adding the test sample.

The methods may then comprise separating the target regions from the first patient from the rest of the sample. In some embodiment the methods comprise conducting a binding moiety pull down assay on the tagged test sample to obtain an enriched sample enriched for amplicons derived from the first patient's target regions. In some embodiments, the binding moiety is attached to a support. In some embodiments the method further comprises attaching the binding moiety to a support and separating the support with attached binding moiety from the unbound nucleic acids. A pull-down assay therefore involves the binding moiety attaching to a support, such that it is immobilized, bound, or captured on the support. The support can then be removed, taking the binding moiety and hybridized nucleic acids with it, or the unbound nucleic acids can be washed away.

Biotin has very high affinity to streptavidin. Therefore, the biotin on the patient-specific oligonucleotide acts as the fusion tag (binding moiety) and immobilized streptavidin acts as its affinity binding ligand (support). In preferred embodiments the support is a streptavidin bead and the binding moiety is a biotin. As is known in the art, if the sample containing biotin is passed over immobilized streptavidin, the biotin and streptavidin will bind, “capturing” any target region bound to or comprising biotin. In some embodiments, binding moiety is Glutathione S-transferase (GST) and the support is Glutathione. In some embodiments, binding moiety is Poly-histidine (polyHis or 6× His) and the support is Nickel or cobalt chelate complexes. Any suitable pull down or purification assay can be used to separate the targets hybridized to the patient-specific oligonucleotides from those that are not.

In some embodiments, the methods then comprise selectively sequencing the enriched sample to determine the sequence of the plurality of target regions from the first patient, optionally thereby identifying the presence or absence of the cancer specific variants.

In some embodiments the methods comprise selectively sequencing the enriched sample to determine the sequence of the plurality of target regions from the first patient, optionally thereby identifying the presence or absence of the cancer specific variants. “Selectively” sequence means preferentially sequencing nucleic acid molecules from or derived from a particular target region from the first patient, i.e. the test patient. Sequencing of nucleic acid molecules from or derived from the other patients regions is avoided by using patient-specific oligonucleotides, comprising a binding moiety, that are specific for the first patient. The sequence of the cancer specific variant is determined during the selective sequencing, as well as determining its presence or absence.

In some embodiments, the method further comprises performing the same hybrid-capture based method for a second, different, patient using test sample from the second patient and patient-specific primers specific to the second patient.

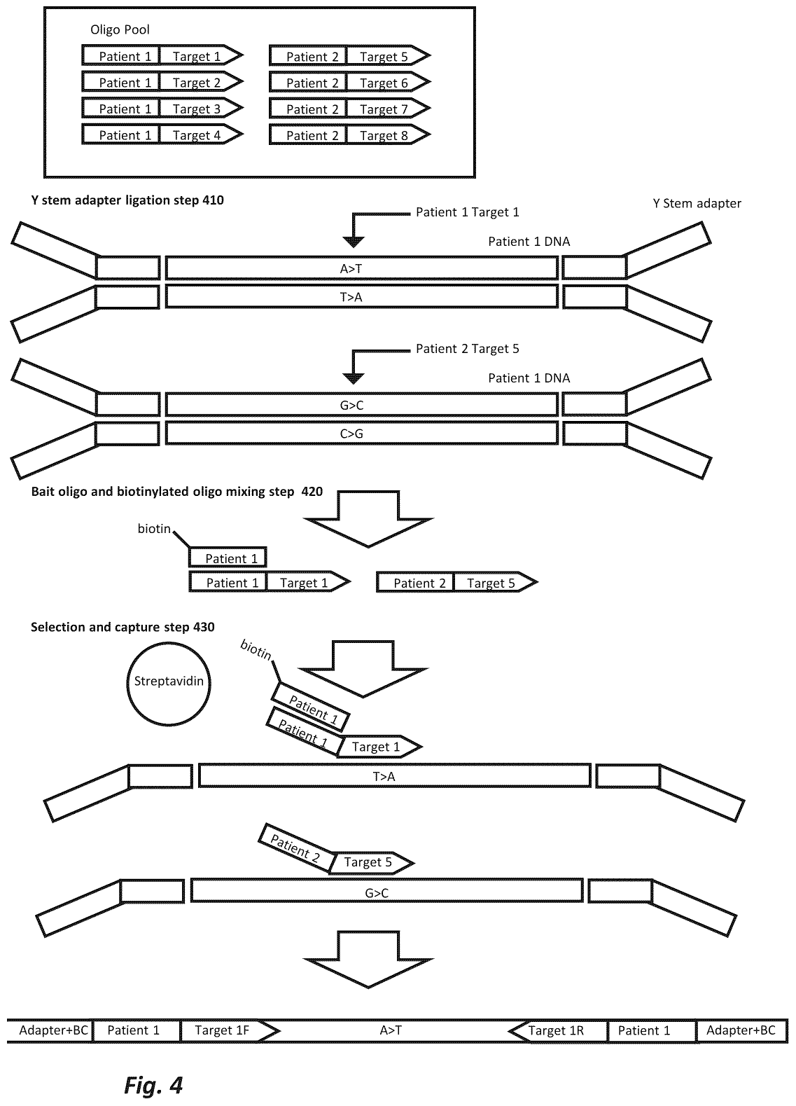

FIG. 4 shows an example embodiment of a selective sequencing method using a hybridization technique as described herein. In this embodiment, “bait” oligonucleotides include a patient-specific identifier (e.g. “Patient 1”) and a target-specific portion (e.g. “Target 3”). For purposes of illustration, only four bait oligonucleotides for patient 1 and four bait oligonucleotides for patient 2 are shown in the “oligo pool”, although a practical oligo pool would include significantly more bait oligonucleotides for both the number of patients and targets, as further described herein. As shown in FIG. 4, a test sample of DNA from patient 1 is first prepared through the ligation 410 of adapters, such as Y-stem adapters comprising two oligonucleotides which have one section with complementary sequence that may bind together, and another section with non-complementary sequence that will not.

A biotinylated oligonucleotide with a sequence complementary to the “patient 1” sequence on the oligonucleotide baits may then be combined 420 with the bait oligonucleotides in the oligo pool. The bait oligonucleotides targeting the regions for patient 1 hybridize to this biotinylated oligonucleotide (including the oligo for patient 1 shown in this example) whilst the other oligonucleotides do not bind (including target 5).

The adapter-ligated DNA from patient 1 may then be contacted 430 with the pool of oligonucleotides and the DNA is allowed to hybridize. A support, e.g. streptavidin is then used to isolate the regions of interest from the test sample of patient 1's DNA, whilst leaving the other regions in the sample.

### Methods as Described Herein for Selective Sequencing of a Plurality of Target Regions Using Synergistic Indirect DNA Hybrid Capture Techniques

In some aspects, synergistic indirect DNA hybridization capture techniques can be used in the general methods described herein. The features and definitions already provided for the general method apply equally to these aspects.

Targeted indirect, synergistic hybridization capture methods can provide more efficient, easy to use, fast, flexible, and practical target nucleic acid capture methods and improved methods for analyzing nucleic acids such as bisulfite treated nucleic acids especially for low-input samples such as cfDNA. Such methods are known in the art, and are described for example, in WO2021155374A2, the contents of which are incorporated herein by reference.

In some aspects are provided methods for selective sequencing of a plurality of target regions from a first patient, each of the target regions containing, or suspected of containing, one or more cancer specific variants, the method comprising providing a pool of bridge oligonucleotides.

Bridge oligonucleotides or “bridge probes” can be used to hybridize a template nucleic acid molecule (for example from a first patient) containing a target region and an adaptor anchor probe. The bridge probe can further allow indirect association between an adaptor anchor probe and target region, facilitating their attachment. The ligation rate of a free adaptor anchor probe and target region can be low because of the randomness of the interaction. But a hybridized bridge probe can increase the probability of ligation between adaptor anchor probe and a target region compared to that with a free adaptor anchor probe. The bridge probe can comprise DNA or RNA. In some embodiments, the bridge probe comprises a first sequence that is complementary to one of the target regions from the first patient; and an identifier sequence specific to the first patient, wherein the identifier sequence is a first adaptor landing sequence.

The bridge probe can comprise a linker connecting the sequence that is complementary to one of the target regions and the identifier sequence. The sequence that is complementary to one of the target regions can be located in the 3′-portion of the bridge probe. The sequence that is complementary to one of the target regions can be located in the 5′-portion of the bridge probe. In some embodiments, the bridge probe can comprise about 400 nucleotides, about 300 nucleotides, about 200 nucleotides, about 120 nucleotides, about 100 nucleotides, about 90 nucleotides, about 80, about 70 nucleotides, about 50 nucleotides, about 40 nucleotides, about 30 nucleotides, about 20 nucleotides, or about 10 nucleotides. In some embodiments, the bridge probe further comprises one or more molecular barcodes. The bridge probe can comprise one or more labels. The bridge probe can comprise one or more binding moieties. The binding moiety can be a biotin. The binding moieties can be attached to a support. The support can be a bead. The bead can be a streptavidin bead.

Multiple bridge probes can be used to anneal to multiple target sequences in each target region. The bridge probes can be designed to have similar melting temperatures. The melting temperatures for a set of bridge probes can be within about 15° C., within about 10° C., within about 5° C., or within about 2° C. The melting temperature for one or more bridge probes can be about 75° C., about 70° C., about 65° C., about 60° C., about 55° C., about 50° C., about 45° C., or about 40° C. The melting temperature for the bridge probe can be about 40° C. to about 75° C., about 45° C. to about 70° C., 45° C. to about 60° C., or about 52° C. to about 58° C.

In some aspects, the pool of oligonucleotides comprises a first sub-population of bridge oligonucleotides, wherein the first sub-population of oligonucleotides comprises at least two classes of bridge probe that target the plurality of target regions from the first patient, wherein the first class of bridge probe comprises:
i. a first sequence that is complementary to one of the target regions from the first patient; and
ii. An identifier sequence specific to the first patient, wherein the identifier sequence is a first adaptor landing sequence capable of hybridizing to a first bridge binding sequence of an anchor probe specific to the first patient,
   and wherein the second class of bridge probe comprises:
iii. a first sequence that is specific to same target region from the first patient as is targeted by the first class of bridge probe but targeting a sequence that flanks (upstream or downstream) the sequence in the target region targeted by the first class of bridge probe; and
iv. an identifier sequence specific to the first patient, wherein the identifier sequence is a second adaptor landing sequence capable of hybridizing to a second bridge binding sequence of the anchor probe specific to the first patient.

The first and second class of bridge probes therefore target the same region, but are complementary to sequences flanking (upstream or downstream) to the target region itself. In order for the patient specific anchor probes to successfully hybridize to the bridge probes, both the first and second class of bridge probe must be hybridized to the target region. If only one bridge probe is hybridized, the patient specific anchor probe will not hybridize to the bridge probe and will not be captured by the support and separated from the other nucleic acids.

In some embodiments the pool of oligonucleotides may also comprise a second (or third or fourth etc.) sub-population of bridge oligonucleotides having the same features as the first sub-population, but targeting a different patient or patients regions. For example, a second sub-population of bridge oligonucleotides, wherein the second sub-population of oligonucleotides comprises at least two classes of bridge probes wherein the first class of bridge probe comprises:
v. a first sequence that is complementary to one of a plurality of target regions from a second patient; and
vi. An identifier sequence specific to the second patient, wherein the identifier sequence is an adaptor landing sequence capable of hybridizing to a first bridge binding sequence of an anchor probe specific to the second patient,
and wherein the second class of bridge probe comprises:
vii. a first sequence that is specific to the same target region from the second patient as is targeted by the first class of bridge probe but targeting a sequence that flanks (upstream or downstream) the sequence in the target region targeted by the first class of bridge probe; and
viii. an identifier sequence specific to the second patient, wherein the identifier sequence is an adaptor landing sequence capable of hybridizing to a second bridge binding sequence of the anchor probe specific to the second patient.

In some embodiments, the methods further comprise contacting a test sample from the first patient with the pool of oligonucleotides.

In some embodiments, the methods further comprise enriching the test sample for nucleic acid molecules from or derived from the first patients target regions using a hybridization capture method by contacting the test sample with patient-specific oligonucleotides, wherein the patient specific oligonucleotides are patient specific anchor probes each comprising a first bridge binding sequence and a second bridge binding sequence that are complementary to the first and second adaptor landing sequences of the patient-specific sequence specific to the first patient.

Patient specific anchor probes bridge binding sequences that hybridize to adaptor landing sequences (identifier sequences) of the one or more bridge probes. The patient specific anchor probes may comprise from 1 to 100 bridge binding sequences. The patient specific anchor probe can comprise spacers in between the bridge binding sequences. The presence of the one or more spacers can improve the efficiency of the hybridization capture and increase the specificity of the capture. In some embodiments the patient specific anchor probes comprise about 400 nucleotides, about 200 nucleotides, about 120 nucleotides, about 100 nucleotides, about 90 nucleotides, about 80 nucleotides, about 70 nucleotides, about 50 nucleotides, about 40 nucleotides, about 30 nucleotides, about 20 nucleotides, or about 10 nucleotides. The adaptor anchor probe can be about 20 to about 70 nucleotides.

In some embodiments the patient specific anchor probes can comprise a molecular barcode. The adaptor anchor probe can comprise an index for distinguishing samples. The molecular barcode or index can be 5′ of the adaptor sequence and 5′ of the bridge binding sequence. The melting temperature of adaptor anchor probe to the bridge probe can be about 65° C., about 60° C., about 55° C., about 50° C., about 45° C. or about 45° C. to about 70° C.

The methods then comprise separating the target regions from the first patient from the rest of the sample. If both the first and second classes of bridge probe have hybridized to regions flanking the target region, the patient specific anchor probe (comprising a binding moiety can hybridize to the bridge probes and be used to separate the target regions. In some embodiment the methods comprise conducting a binding moiety pull down assay on the tagged test sample to obtain an enriched sample enriched for amplicons derived from the first patient's target regions. In some embodiments, the binding moiety is attached to a support. In some embodiments the methods further comprises attaching the binding moiety to a support and separating the support with attached binding moiety from the unbound nucleic acids. A pull down assay therefore involves the binding moiety attaching to a support, such that it is immobilized, bound or captured on the support. The support can then be removed, taking the binding moiety and hybridized nucleic acids with it, or the unbound nucleic acids can be washed away.

In some embodiments, the patient specific anchor probes can comprise a binding moiety. In preferred embodiments the binding moiety is biotin. In some embodiments, the binding moieties can be attached to a support. The support can be a bead. The bead can be a streptavidin bead. The patient specific anchor probes can comprise a label, such as a fluorescent or radioactive label.

In some embodiments, the binding moiety is Glutathione S-transferase (GST) and the support is Glutathione. In some embodiments, binding moiety is Poly-histidine (polyHis or 6× His) and the support is Nickel or cobalt chelate complexes.

The methods comprise selectively sequencing the enriched sample to determine the sequence of the plurality of target regions from the first patient, optionally thereby identifying the presence or absence of the cancer specific variants. “Selectively” sequence means preferentially sequencing nucleic acid molecules from or derived from the first patient's target regions, i.e. the test patient. Sequencing of nucleic acid molecules from or derived from the other patient's target regions is avoided by using patient-specific anchor probes, comprising a binding moiety, that are specific for the first patient. The sequence of the cancer specific variant is determined during the selective sequencing, as well as determining its presence or absence.

In some embodiments, the method further comprises performing the same indirect hybrid-capture based method for a second, different, patient using test sample from the second patient and patient-specific anchor probes specific to the second patient.

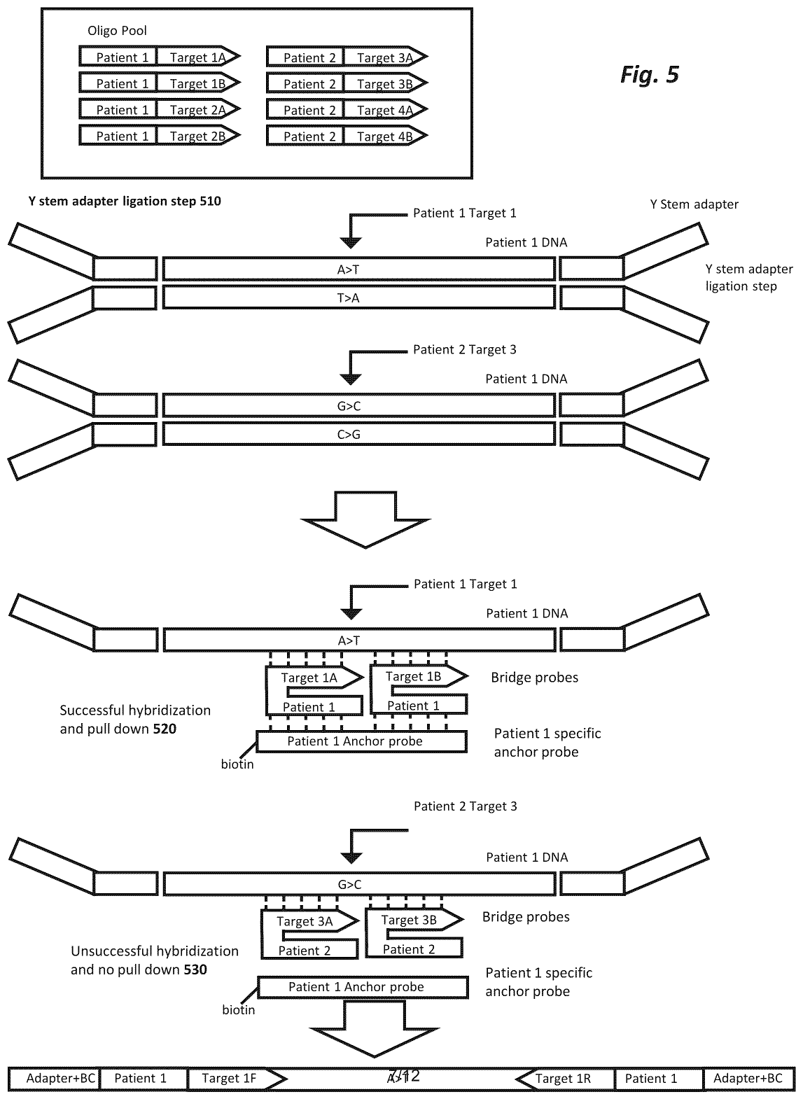

FIG. 5 shows an example embodiment of a selective sequencing method using synergistic indirect DNA hybrid capture techniques as described herein. In this embodiment, “bridge probe” oligonucleotides can include a patient-specific identifier (e.g. “Patient 1”) and a target-specific portion (e.g. “Target 1A”). Like in the other examples, two pairs of bridge probes for patient 1 and two pairs of bridge probes for patient 2 are shown in the “oligo pool”, although a practical oligo pool would include significantly more bridge probes as further described herein.

First, a test sample of DNA from patient 1 is prepared through the ligation 510 of adapters, such as Y-Stem adapters. This patient specific DNA is combined with both 1) the oligo pool of bridge probes and 2) a biotinylated anchor probe that has a first bridge binding sequence and a second bridge binding sequence, both which are complementary to the patient 1 specific sequence on the bridge probes. In this example the bridge probes complementary to patient 1's target 1 (target 1A and target 1B) and patient 2's target 3 are shown, but all bridge probes would be present as the test sample of DNA has been contacted with the oligo pool. An anchor probe complementary to the patient 1 identifier sequence is then contacted with the sample. The patient 1 bridge probes bind 520 the patient 1 anchor probe and the patient 1 DNA allowing effective pull down 520; however, the patient 2 bridge probes do not bind 530 the patient 1 anchor probe. Streptavidin may then be used to select and pull down the regions of interest solely from patient 1's DNA whilst leaving the other regions.

### Methods as Described Herein for Selective Sequencing of a Plurality of Target Regions Using Oligonucleotide Pool Enrichment Techniques

In some embodiments, the pool of oligonucleotides may first be enriched for patient-specific sequences prior to being contacted with a test sample, thereby creating a functional targeting reagent.

The term “enrich” or “enrichment” as used herein refers to processes that increase the relative abundance of a specific nucleotide sequence within a complex mixture. Enrichment can make it easier to detect, target, identify, or otherwise use a specific nucleotide sequence. In some embodiments, a test sample of DNA is enriched for certain sequences by contacting the test sample of DNA with a pool of oligonucleotides targeting those sequences. Various methods to enrich a sequence are known to the skilled person, for example, PCR, chromatography, immunoprecipitation, size selection, exome capture and hybridization based enrichment. For example, a test sample may be enriched for amplicons derived from a first patient's target regions by contacting the test sample with primer pairs specific for the first patient and performing PCR, increasing the relative abundance of amplicons derived from a first patient's target regions. In other embodiments, a pool of oligonucleotides may first be enriched, for example, by functionalizing specific nucleotide sequences within the pool (e.g. by biotinylation), therefore allowing the pool to enrich for only patient-specific sequences. A pool may also be enriched by removing or degrading some or all nucleotide sequences within the pool except for specific nucleotide sequences, for example by enzyme digestion, therefore similarly allowing the pool to enrich for only patient-specific sequences.

Accordingly, in a seventh aspect is provided a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. enriching the pool of oligonucleotides for those oligonucleotides containing the identifier sequence specific to the first patient;
c. contacting a test sample from the first patient with the enriched pool of oligonucleotides;
d. selectively sequencing the plurality of target regions from the first patient, optionally thereby identifying the presence or absence of the cancer specific variants.

In certain embodiments of the seventh aspect is provided a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. enriching the pool of oligonucleotides for the first sub-population of oligonucleotides, wherein the enriching comprises contacting the pool of oligonucleotides with oligonucleotides each comprising a sequence that is complementary to the identifier sequence specific to the first patient;
c. contacting a test sample from the first patient with the enriched pool of oligonucleotides; and
d. selectively sequencing the plurality of target regions from the first patient, optionally thereby identifying the presence or absence of the cancer specific variants.

In some embodiments the step of enriching the pool further comprises extending and copying the first sub-population of oligonucleotides.

In some embodiments, the step of enriching the pool further comprises:
e. contacting the pool of oligonucleotides with forward and reverse primers complementary to the patient-specific sequence of the first sub-population of oligonucleotides; and
f. amplifying the first sub-population using the forward and reverse primers, thereby enriching for the first sub-population.

In some embodiments the method further comprises degrading or removing the second sub-population of oligonucleotides from the pool of oligonucleotides prior to contacting the pool with the test sample.

In some embodiments the oligonucleotides comprising a sequence that is complementary to the identifier sequence specific to the first patient further comprises a biotin.

In certain embodiments, the patient-specific sequence further comprises a first patient-specific sequence and a second patient-specific sequence.

In certain embodiments of the seventh aspect is provided a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and a first identifier sequence specific to the first patient and a second identifier sequence specific to the first patient;
   ii. a second sub-population of oligonucleotides, wherein each member of the second sub-population of oligonucleotides comprises a first sequence that is complementary to one of the plurality of target regions from a second patient and a first identifier sequence specific to the second patient and a second identifier sequence specific to the second patient;
b. enriching the pool of oligonucleotides for the first sub-population of oligonucleotides, wherein the enriching comprises contacting the pool of oligonucleotides with oligonucleotides each comprising a sequence that is complementary to the identifier sequence specific to the first patient;
c. contacting a test sample from the first patient with the enriched pool of oligonucleotides; and
d. selectively sequencing the plurality of target regions from the first patient, optionally thereby identifying the presence or absence of the cancer specific variants.

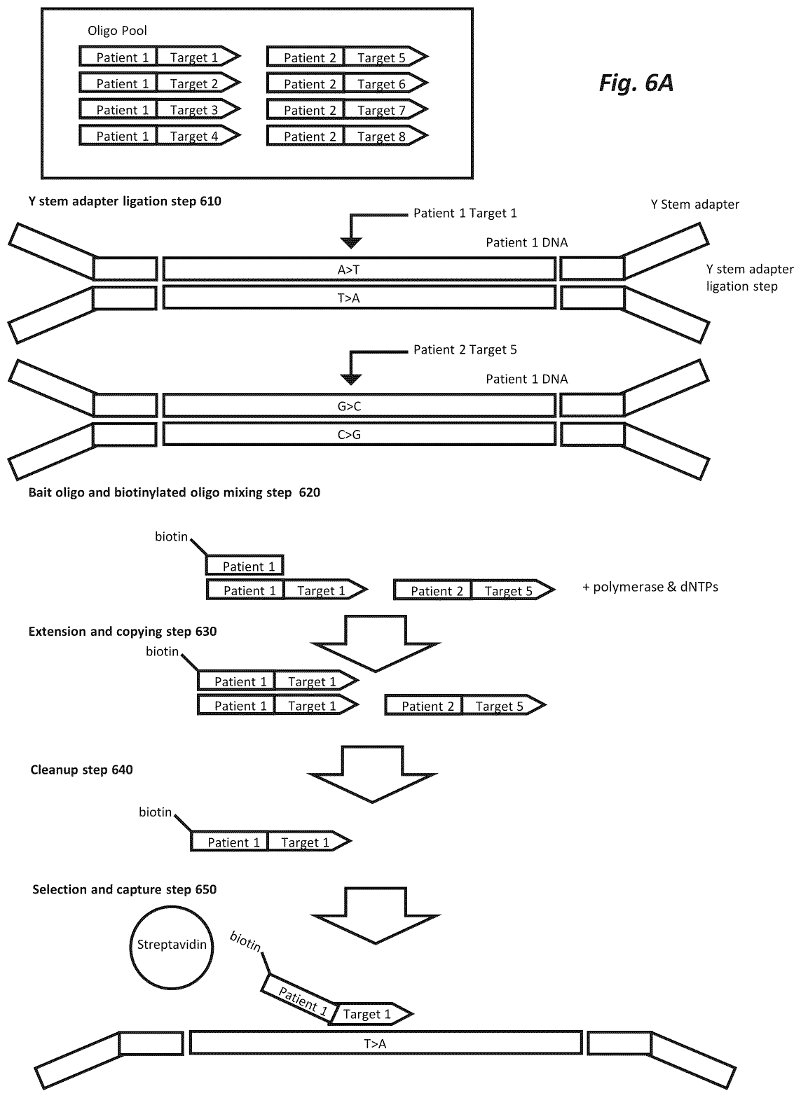

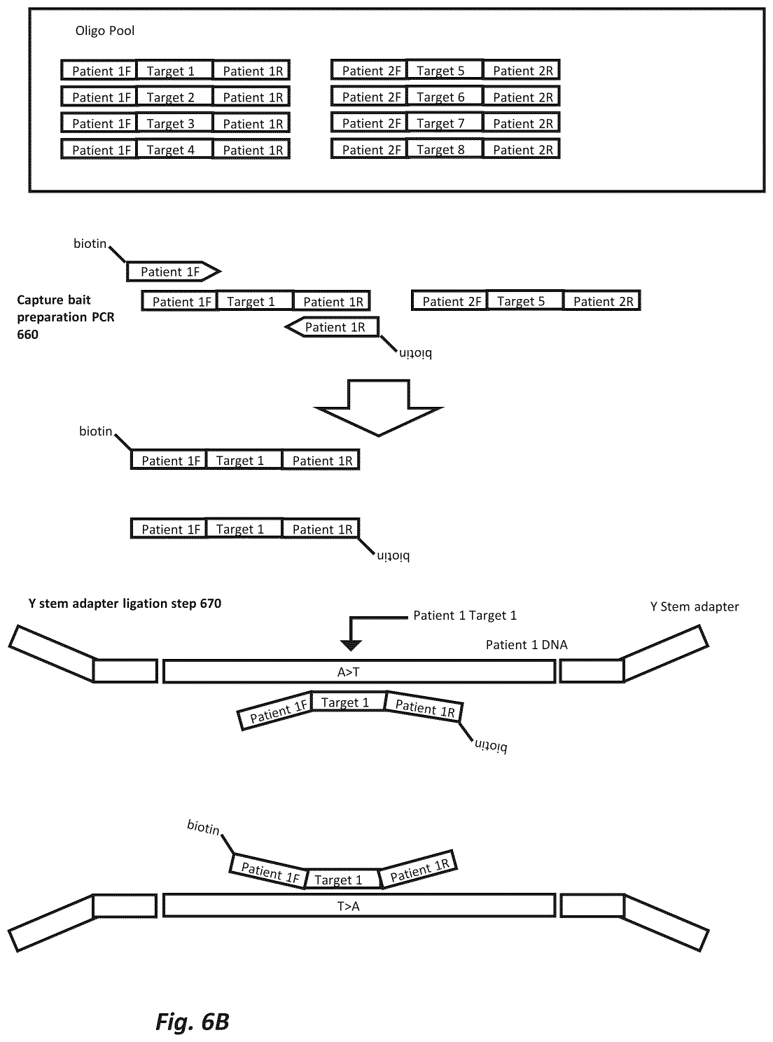

For example, in some embodiments, a patient-specific oligonucleotide comprising a binding moiety such as biotin may first be combined with the bait oligonucleotides through hybridization at the identifier sequence. An enzyme such as a polymerase is then used to enable the patient-specific oligonucleotide to copy the target specific region of the bait oligonucleotides. Optionally the original bait oligonucleotides are degraded, for example, through cleavage of uracil bases added to the bait oligonucleotides or are cleaned away using the binding moiety. The remaining binding moiety tagged molecules are then applied to the test sample and used for hybrid capture. As shown in further detail in FIG. 6A, such “bait” oligonucleotides may include a patient-specific identifier (e.g. “Patient 1”) and a target-specific portion (e.g. “Target 2”). As in the other examples, four bait oligonucleotides for patient 1 and four bait oligonucleotides for patient 2 are shown in the “oligo pool”, although a practical oligo pool would include significantly more bait oligonucleotides, as further described herein. First, a test sample of DNA from patient 1 is prepared through the ligation 610 of adapters, as previously described.

As further shown in FIG. 6A, a biotinylated oligonucleotide with a sequence complementary to the “patient 1” sequence on the oligonucleotide baits may be combined 620 with the bait oligonucleotides in the pool of oligonucleotides. The bait oligonucleotides targeting the regions for patient 1 hybridize to this biotinylated oligonucleotide (including the oligo for patient 1 shown in this example) whilst the other oligonucleotides do not bind (including target 5). A polymerase and dNTPS may then be added. This causes the biotinylated oligonucleotide to be extended, copying 630 the target specific sequence of the original oligos but only when the original oligo in the pool has a patient 1 sequence. The unbiotinylated oligos are then optionally either removed 640 by denaturing the DNA then pulling down the tagged molecule (e.g. with Streptavidin) or through selective degradation (e.g. through uracils in the original molecule). In this way, the oligo pool is enriched for only bait oligonucleotides containing patient 1 sequence. It would be understood that step 620 and step 640 could equally be achieved by using biotinylated dNTPs (deoxynucleotide triphosphate) than by adding biotin to the oligonucleotide.

The adapter-ligated DNA from patient 1 may be contacted 650 with this remaining pool of oligonucleotides and the DNA is allowed to hybridize. Streptavidin may then be used to select the regions of interest from patient 1's DNA whilst leaving the other regions.

In some embodiments, the oligonucleotide pool may be enriched for patient-specific sequences using PCR. As shown in FIG. 6B, a pair of biotinylated PCR primers (oligonucleotides) each with a sequence targeting the “patient 1” sequences on the oligonucleotides baits may be combined 660 with the bait oligonucleotides. The forward primer matches the sequences on the bait oligonucleotides whilst the reverse primer has the reverse complement of its target sequence. The bait oligonucleotides targeting the regions for patient 1 hybridize to these biotinylated primers (including the oligo for patient 1 shown in this example) whilst the other oligonucleotides do not bind (including target 5). A polymerase and dNTPS are also added. Temperatures are then cycled to enable PCR. This causes the biotinylated primers to be extended, copying the target specific sequence of the original oligos but only when the original oligo in the pool has patient 1 sequences, thus creating an enriched pool of oligonucleotides largely containing those oligonucleotides with patient 1 sequences. The unbiotinylated oligos may then optionally be either removed by denaturing the DNA then pulling down the biotinylated molecule (e.g. with Streptavidin) or through selective degradation (e.g. through uracils in the original molecule) (not shown).

A test sample of adapter-ligated DNA from patient 1 may then be contacted 670 with the enriched pool of oligonucleotides and the DNA is allowed to hybridize. A support, e.g. streptavidin may then be used to select the regions of interest from patient 1's DNA whilst leaving the other regions, thereby enriching the test sample for those targets of interest in patient 1.

### Methods as Described Herein for Selective Sequencing of a Plurality of Target Regions Using Selective Cleavage or Cutting of Patient Specific Sequence Present in Oligonucleotides

Nucleotide sequences can be cleaved, cut or degraded in a number of ways. Some common methods of cleaving nucleotide sequences, such as DNA sequences, include enzymatic cleavage using enzymes such as endonucleases, exonucleases or restriction enzymes; use of specific nucleases such as TALENS, Zinc-finger nucleases, or RNA guided nucleases; CRISPR technology; chemical cleavage; mechanical cleavage or shearing; ultrasonication; or electroporation.

Cleaving agents as described herein can therefore be selected from the group consisting of endonucleases, exonucleases, restriction enzymes; nucleases such as TALENS, zinc-finger nucleases, or RNA guided nucleases, CRISPR-Cas complexes; chemical cleavage agents and mechanical cleavage agents.

Restriction enzymes are enzymes that recognize specific nucleotide sequences e.g. DNA sequences and catalyze the cleavage of the nucleotide e.g. DNA at or near these recognition sites. These enzymes are widely used in molecular biology for tasks like DNA cloning, restriction mapping, and creating DNA fragments with defined ends. Restriction enzyme sites are the sequences that are recognized by a particular restriction enzyme, and one or more restriction enzyme sites can be incorporated into a nucleotide sequence to enable it to be cleaved or digested by restriction enzymes in specific places.

Some enzymes used to cut DNA do not recognize specific nucleotide sequences but cleave DNA at random sites. For example, certain exonucleases, such as exonuclease III, can cleave DNA from one end, degrading it sequentially. Certain endonucleases, such as DNase I, cleaves DNA at random phosphodiester bonds in the backbone.

CRISPR (Clustered Regularly Interspaced Short Palindromic Repeats) is a gene-editing technology that allows DNA to be precisely modified. CRISPR systems comprise two components: CRISPR arrays and CRISPR-associated (Cas) proteins. CRISPR arrays are specific DNA sequences based on those found in the genomes of bacteria and archaea that contain short, repetitive sequences interspersed with unique “spacer” sequences derived from past viral or plasmid invasions. Cas proteins, such as Cas9, are enzymes that work in connection with CRISPR arrays by recognizing and cleaving DNA sequences that match the spacer sequences in the CRISPR arrays. CRISPR systems can be used to edit nucleotide sequences by designing synthetic RNA molecule, called a guide RNA (gRNA), with a sequence that matches the target DNA to be edited. The gRNA is combined with a Cas protein (such as Cas9), which binds to the gRNA and guides it to the complementary DNA sequence in the target DNA sample. The Cas protein then cleaves the DNA at that precise location. The resulting break in the DNA can then be repaired by the cell's natural DNA repair mechanisms, which can introduce specific mutations into the DNA at the site of the cut. A DNA template containing specific sequences or mutations can also be supplied during the repair process to introduce said sequences or mutations to the target DNA.

TAL effector nucleases (TALENs) are similar to the CRISPR-Cas system, and are proteins that recognize specific DNA sequences and introduce double-strand breaks at those sequences. Zinc finger nucleases are also engineered proteins that can be designed to target specific DNA sequences and cleave them. They comprise a zinc finger proteins (naturally occurring DNA-binding proteins that can be engineered to recognize specific DNA sequences) and a nuclease domain that cleaves the DNA at or near the specific sequence recognized by the zinc finger protein.

Chemical cleavage refers to the use of chemical agents, such as hydroxyl radicals, osmium tetroxide, piperidine, bleomycin and other agents to cleave DNA.

Mechanical cleavage or shearing refers to mechanical (as opposed to chemical) processes of cleaving DNA, such as nebulization (using compressed gas to shear DNA into fragments) or ultrasonication (employing high-frequency sound waves to mechanically disrupt DNA strands).

The choice of method for nucleotide cleavage depends on factors such as the specificity required, the location of the desired cleavage site, and the intended applications.

In certain embodiments of the seventh aspect is provided a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and a first identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. enriching the pool of oligonucleotides for the first sub-population of oligonucleotides, wherein the enriching comprises contacting the pool of oligonucleotides with a cleaving agent that targets a cut site present in the patient-specific sequence of the second sub-population of oligonucleotides;
c. contacting a test sample from the first patient with the enriched pool of oligonucleotides; and
d. selectively sequencing the plurality of target regions from the first patient, optionally thereby identifying the presence or absence of the cancer specific variants.

In certain other embodiments of the seventh aspect is provided a method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
   i. a first sub-population of oligonucleotides that target the plurality of target regions from the first patient, wherein each member of the first sub-population of oligonucleotides comprises a first sequence that is complementary to one of the plurality of target regions from the first patient and a first identifier sequence specific to the first patient;
   ii. a second sub-population of oligonucleotides, wherein each member of the second sub-population of oligonucleotides comprises a first sequence that is complementary to one of the plurality of target regions from a second patient and an identifier sequence specific to the second patient;
b. enriching the pool of oligonucleotides for the first sub-population of oligonucleotides, wherein the enriching comprises contacting the pool of oligonucleotides with a cleaving agent that targets a cut site present in the patient-specific sequence of the first sub-population of oligonucleotides;
c. contacting a test sample from the first patient with the enriched pool of oligonucleotides; and
d. selectively sequencing the plurality of target regions from the first patient, optionally thereby identifying the presence or absence of the cancer specific variants.

In some embodiments the cleaving agents may be selected from the group consisting of endonucleases, exonucleases, restriction enzymes; nucleases such as TALENS, zinc-finger nucleases, or RNA guided nucleases, CRISPR-Cas complexes; chemical cleavage agents and mechanical cleavage agents.

In some embodiments the oligonucleotides in the pool of oligonucleotides further comprise a binding moiety. In some embodiments, contacting a test sample from the first patient with the enriched pool of oligonucleotides further comprises contacting the test sample with streptavidin beads. In some embodiments the oligonucleotides in the pool of oligonucleotides are bound to a solid support. In some embodiments, contacting the pool of oligonucleotides with a cleaving agent further comprises releasing the first sub-population of oligonucleotides from the solid support. In some embodiments, the solid support is a streptavidin bead.

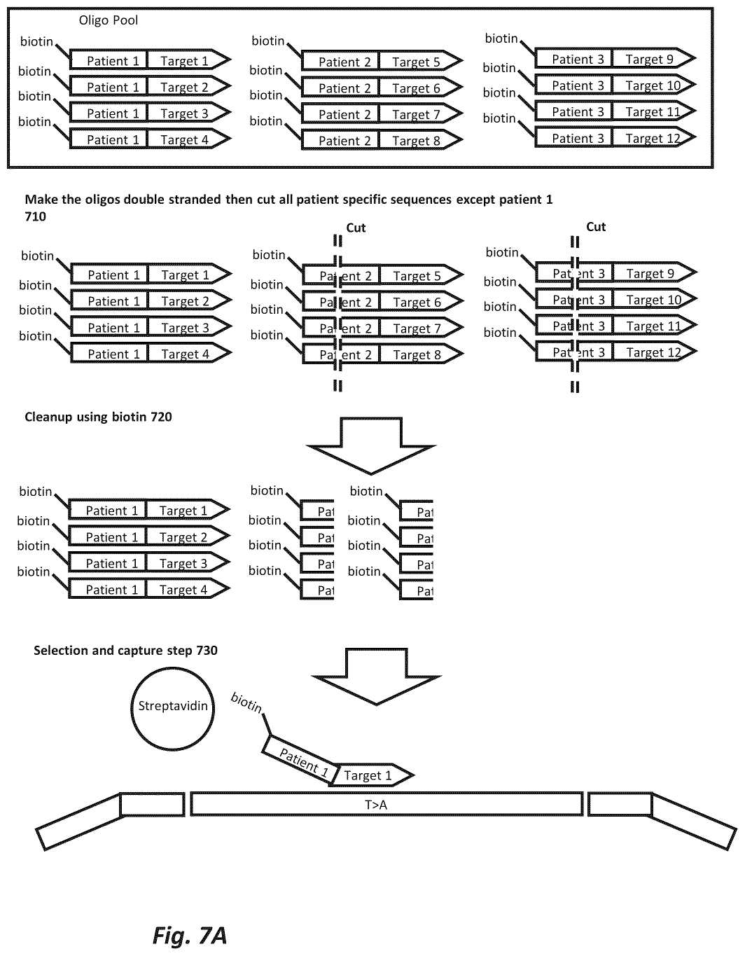

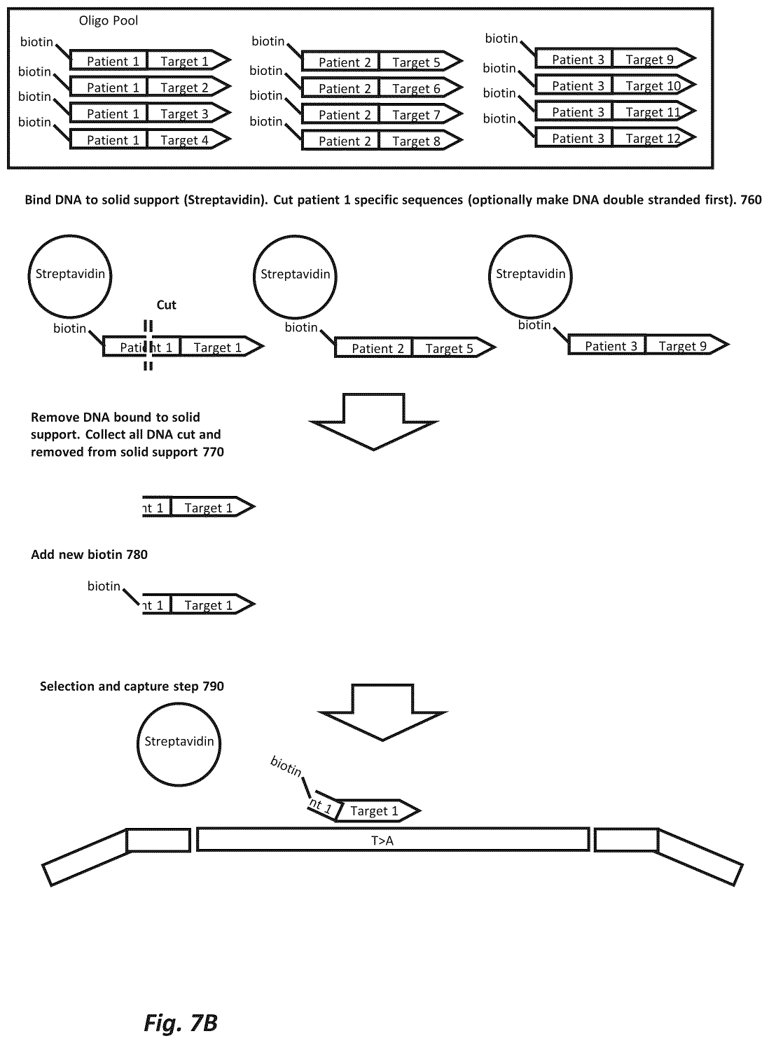

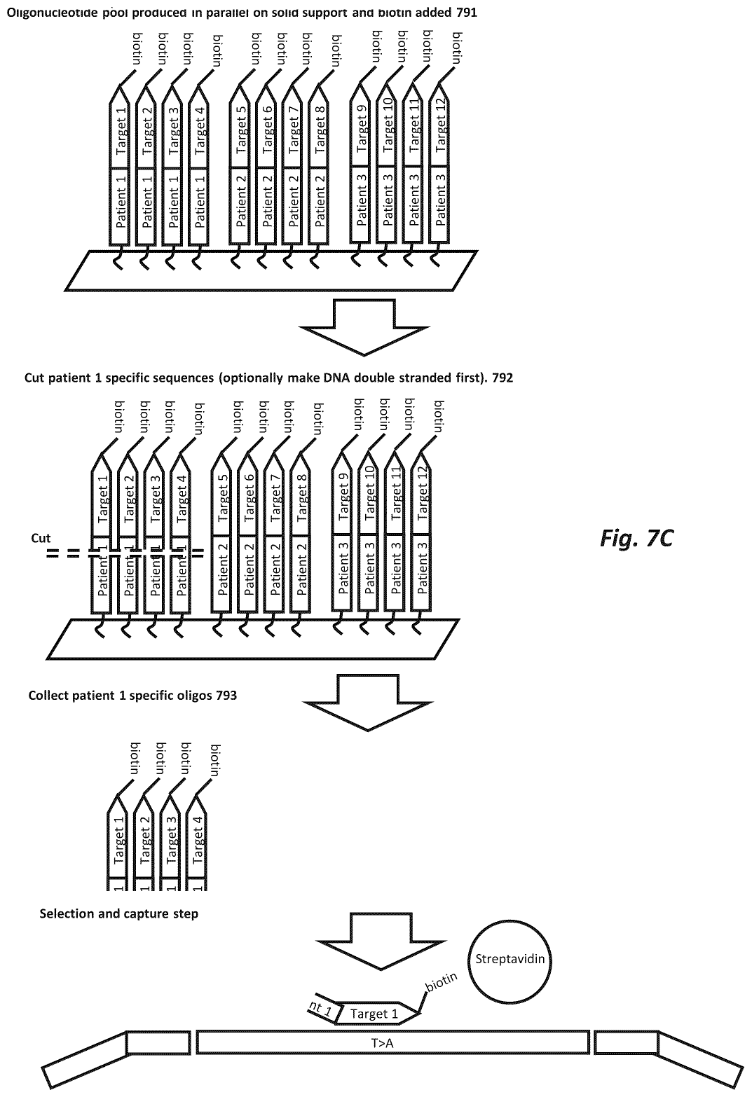

In some embodiments, restriction enzymes or other DNA cutting or cleaving agents may be used to cleave or cut certain DNA sequences in an oligo pool according to the disclosure, thereby enriching for patient specific sequence. FIG. 7A shows an example embodiment of a selective sequencing method using a hybridization technique and a DNA cleavage approach as described herein. In this embodiment, “bait” oligonucleotides include a patient-specific identifier (e.g. “Patient 1”) with a binding moiety (e.g., a biotin) at one end and a target-specific portion (e.g. “Target 1”) at the opposite end. For purposes of illustration, only four bait oligonucleotides for patients 1, 2 and 3 are shown in the “oligo pool”, although a practical oligo pool would include significantly more bait oligonucleotides for both the number of patients and targets, as further described herein. As shown in FIG. 7A, first the oligos are made double stranded at least in the patient specific regions. This may be achieved in multiple ways, but a simple method is mixing the oligo pool with another pool of oligos that contains the reverse complement of the patient specific sequences. All patient specific sequences except that of patient 1's may then be cut 710 using, e.g., CAS9 and guide RNAs targeting all sequences expect that of patient 1s. This removes the biotin from all oligos except those belonging to patient 1. The patient 1 specific oligos may then be cleaned up using, e.g., streptavidin beads are used to capture the patient 1 specific oligos that comprise the binding moiety e.g. biotin.

DNA from patient 1 is then prepared through the ligation of adapters, such as Y-stem adapters comprising two oligonucleotides which have one section with complementary sequence that may bind together, and another section with non-complementary sequence that will not.

The adapter-ligated DNA from patient 1 may then be contacted with the pool of oligonucleotides and the DNA is allowed to hybridize. Streptavidin is then used to isolate the regions of interest from patient 1's DNA, whilst leaving the other regions in the sample.

Note, whilst in this and the methods below involving cutting of DNA, it may be preferable to make the Oligo pool double stranded as some methods only cut double stranded DNA however there are methods that also cut single stranded DNA.

FIG. 7B shows another example embodiment of a selective sequencing method using a hybridization technique and a DNA cleavage approach as described herein. In this embodiment, “bait” oligonucleotides include a patient-specific identifier (e.g. “Patient 1”) with a biotin at its end and a target-specific portion (e.g. “Target A”) at the opposite end. For purposes of illustration, only four bait oligonucleotides for patients 1, 2 and 3 are shown in the “oligo pool”, although a practical oligo pool would include significantly more bait oligonucleotides for both the number of patients and targets, as further described herein. As shown, first the oligos are optionally made double stranded at least in the patient specific regions. This may be achieved in multiple ways but a simple method is mixing the oligo pool with another pool of oligos that contains the reverse complement of the patient specific sequences. The oligos are then combined with a support, such as streptavidin coated magnetic beads. The patient 1 specific sequence is then cut 760 using (e.g.) CAS9 and a guide RNA targeting this patient 1 specific sequence. This effectively releases the patient 1 oligos whilst leaving all other oligos attached to the support. Magnetic beads are used to remove 770 all other oligonucleotides, as these other oligonucleotides have not been detached from their solid support. The patient 1 oligos in the subsequent pool are then attached 780 to a new biotin molecule.

As the patient 1 oligos have been removed from the initial pool, the initial pool may still contain oligos for other patients. Accordingly, this process may then be repeated releasing the other patients oligos in a sequential fashion, thereby making full use of the oligo pool.

DNA from patient 1 may then be prepared through the ligation of adapters, such as Y-stem adapters comprising two oligonucleotides which have one section with complementary sequence that may bind together, and another section with non-complementary sequence that will not.

The adapter-ligated DNA from patient 1 may then be contacted 790 with the pool of oligonucleotides and the DNA is allowed to hybridize. A support, e.g., streptavidin may then be used to isolate the regions of interest from patient 1's DNA, whilst leaving the other regions in the sample.

FIG. 7C shows another example embodiment of a selective sequencing method using a hybridization technique as described herein. In this embodiment, the “bait” oligonucleotides are synthesized 791 in parallel on a solid support (e.g., a silicon wafer). All oligonucleotides include a patient-specific identifier (e.g. “Patient 1”) and a target specific sequence (e.g. “Target 1”), which is attached to a binding moiety (e.g., a biotin) at one end and to the solid support at the opposite end. For purposes of illustration, only four bait oligonucleotides for patients 1, 2 and 3 are shown in the “oligo pool”, although a practical oligo pool would include significantly more bait oligonucleotides for both the number of patients and targets, as further described herein. As shown, first the oligos are optionally made double stranded at least in the patient specific regions. This may be achieved in multiple ways but a simple method is mixing the oligo pool with another pool of oligos that contains the reverse compliment of the patient specific sequences. The oligos are then combined with streptavidin coated magnetic beads. The patient 1 specific sequence may then be cut 792 using (e.g.) CAS9 and a guide RNA targeting this sequence. This effectively releases the patient 1 oligos whilst leaving all other oligos attached to the solid support.

The patient 1 oligos may then be collected or otherwise separated 793 from the oligo pool. Similar to FIG. 7B, this process may then be repeated releasing the other patients oligos in a sequential fashion.

DNA from patient 1 is then prepared through the ligation of adapters, such as Y-stem adapters comprising two oligonucleotides which have one section with complementary sequence that may bind together, and another section with non-complementary sequence that will not.

The adapter-ligated DNA from patient 1 may then be contacted 794 with the pool of oligonucleotides and the DNA is allowed to hybridize. Streptavidin is then used to isolate the regions of interest from patient 1's DNA, whilst leaving the other regions in the sample.

In some embodiments, the oligonucleotides containing patient specific sequences designed to be cut in a specific way may first be amplified by rolling circle amplification. In this and other embodiments described, some of the nucleotides used in the amplification may be biotinylated nucleotides. Following amplification, the DNA fragments would be cut at just the one patient specific sequence. Optionally an additional step would then be performed that would select just the short DNA molecules (e.g. those less than 1000 or less than 500 or less than 200 bp).

### Target Regions and Variants

These embodiments apply to any of the aspects described above.

In some embodiments of any of the aspects described herein, the plurality of target regions from a first patient are genomic target regions. The number of target regions from the first and/or second patient may be at least about 100. The number of target regions from the first and/or second patient may be from about 100 to about 50,000 target regions. In some preferred embodiments, number of target regions from the first and/or second patient is from about 1000 to about 50,000 target regions.

In some embodiments of any of the aspects described herein, the one or more cancer specific variants were previously identified in a cancer from the first patient. The one or more cancer specific variants may be known cancer specific variants.

In some embodiments of any of the aspects described herein, the patient has or has previously had cancer.

In some embodiments of any of the aspects described herein, the method comprises, before step (a), identifying one or more cancer specific variants that are present within the patient's cancer. The methods may comprise identifying 100 or more cancer specific variants that are present within the patient's cancer, or identifying 1000 or more cancer specific variants that are present within the patient's cancer. In any embodiment, the methods may comprise identifying from 10 to 1,000,000 cancer specific variants, or from 100 to 100,000 cancer specific variants, or from 500 to 50,000 cancer specific variants that are present within a patient's cancer, or from 1,000 to 10,000 cancer specific variants, or from 1,000 to 20,000 cancer specific variants. In some embodiments, the cancer-specific variants are identified from a tumor tissue sample from the patient.

### Further Method Steps

These embodiments apply to any of the aspects described above.

In some embodiments of any of the aspects described herein, the methods comprise sequencing of nucleic acid molecules from or derived from the first patient and does not comprise sequencing of nucleic acid molecules from or derived from the second (or any other) patient.

In some embodiments of any of the aspects described herein, the methods further comprise performing the same method for the second patient using a test sample from a second patient and patient-specific oligonucleotides specific to the second patient. For example, in some embodiments the methods may further comprise:
e. contacting a test sample from the second patient with the pool of oligonucleotides;
f. contacting the test sample with patient-specific oligonucleotides each comprising a sequence that is complementary to the patient-specific sequence specific to the second patient;
g. using the patient-specific oligonucleotides, selectively sequencing the plurality of target regions from the second patient, optionally thereby identifying the presence or absence of the cancer specific variants.

In some embodiments of any of the aspects described herein, each oligonucleotide in the first sub-population of oligonucleotides comprises the same patient-specific sequence specific to the first patient, or wherein each oligonucleotide in the first sub-population of oligonucleotides comprises either the same patient-specific sequence specific to the first patient or the reverse complement thereof. The pool of oligonucleotides may further comprise N additional sub-populations of oligonucleotides, each additional sub-population specific for another patient. In some embodiments, the pool of oligonucleotides further comprises N additional patient-specific sub-populations of oligonucleotides, wherein each member of each patient-specific sub-population of oligonucleotides comprises a first sequence that is complementary to one of a plurality of target regions from that sub-population's patient, and a patient-specific sequence specific to that sub-population's patient, wherein N is from 1 to 50, or N is from 1 to 200, or N is from 1 to 1000.

### Downstream Analysis of Enriched Samples

The enriched samples generated using methods described herein can be further analyzed using various methods including southern blotting, polymerase chain reaction (PCR) (e.g., real-time PCR (RT-PCR), digital PCR (dPCR), droplet digital PCR (ddPCR), quantitative PCR (Q-PCR), nCounter analysis (Nanostring technology), gel electrophoresis, DNA microarray, mass spectrometry (e.g., tandem mass spectrometry, matrix-assisted laser desorption ionization time of flight mass spectrometry (MALDI-TOF MS), chain termination sequencing (Sanger sequencing), or next generation sequencing.

In some embodiments, the presence or absence of the cancer specific variants may be determined using digital PCR (dPCR), droplet digital PCR (ddPCR), quantitative PCR (Q-PCR).

The next generation sequencing can comprise 454 sequencing (ROCHE) (using pyrosequencing), sequencing using reversible terminator dyes (ILLUMINA sequencing), semiconductor sequencing (THERMOFISHER ION TORRENT), single molecule real time (SMRT) sequencing (PACIFIC BIOSCIENCES), nanopore sequencing (e.g., using technology from OXFORD NANOPORE or GENIA), microdroplet single molecule sequencing using pyrophosphorolyis (BASE4), single molecule electronic detection sequencing, e.g., measuring tunnel current through nanoelectrodes as nucleic acid (DNA/RNA) passes through nanogaps and calculating the current difference (QUANTUM SEQUENCING from QUANTUM BIOSYSTEMS), GenapSys Gene Electronic Nano-Integrated Ultra-Sensitive (GENIUS) technology (GENAPYS), GENEREADER from QIAGEN, sequencing using sequential hybridization and ligation of partially random oligonucleotides with a central determined base (or pair of bases) identified by a specific fluorophore (SOLID sequencing) or sequencing by binding (PacBio Onso). The sequencing can be paired-end sequencing.

The number of target sequences from a sample that can be sequenced using methods described herein can be about 5, 10, 15, 25, 50, 100, 1000, 10,000, 100,000, or 1,000,000, or about 5 to about 100, about 100 to about 1000, about 1000 to about 10,000, about 10,000 to about 100,000, or about 100,000 to about 1,000,000.

The sequencing can generate at least 100, 1000, 5000, 10,000, 100,000, 1,000,000, or 10,000,000 sequence reads per target region. The sequencing can generate between about 100 sequence reads to about 1000 sequence reads, between about 1000 sequence reads to about 10,000 sequence reads, between about 10,000 sequence reads to about 100,000sequence reads, between about 100,000 sequence reads and about 1,000,000 sequence reads, or between about 1,000,000 sequence reads and about 10,000,000 sequence reads.

The depth of sequencing can be about 1×, 5×, 10×, 50×, 100×, 1000×, 10,000×, or 100,000×. The depth of sequencing can be between about 1× and about 10×, between about 10× and about 100×, between about 100× and about 1000×, between about 1000× and about 10,000× or between about 1000× and about 100,000×.

In some embodiments of any of the aspects described herein, the methods further comprise identifying the presence or absence of the cancer-specific variants. Identifying the presence or absence of cancer-specific variants can comprise comparing the number of sequence reads supporting the presence of each cancer-specific variant to an error probability distribution, as further described in International Patent Publication WO 2023-012521, the contents of which are hereby incorporated by reference in their entirety

In some embodiments of any of the aspects described herein, the methods further comprise identifying cancer DNA in the test sample. In some embodiments, identifying cancer DNA in the test sample can comprise combining evidence from each cancer-specific variant (e.g., a metric based on the number of sequence reads supporting the presence or absence of each cancer-specific variant) and then determining whether cancer DNA is present in the test sample, as further described in International Patent Publication WO 2023-012521.

### Test Samples

In some embodiments, the “test sample”, “patient sample” or “sample” is a biological sample obtained from a subject, or a sample that is extracted from a biological sample obtained from a subject. The patient sample can be a tissue sample, for example a surgical sample. Preferably the sample is a liquid biopsy sample, such as blood, plasma, serum, urine, seminal fluid, stool, sputum, pleural fluid, ascetic fluid, synovial fluid, cerebrospinal fluid, lymph, nipple fluid, cyst fluid, or bronchial lavage. In some embodiments the sample is a cytological sample or smear or a fluid containing cellular material, such as cervical smear, nasal brushing, esophageal sampling by a sponge (cytosponge), endoscopic/gastroscopic/colonoscopic biopsy or brushing, cervical mucus or brushing. In a preferred embodiment, the test sample is a blood sample.

Many of the above samples can be obtained non-invasively, and can therefore be taken regularly without great risk or discomfort to the subject. Methods disclosed herein may comprise a step of obtaining a sample from a patient. Alternatively, the methods may be carried out on samples previously obtained from a patient (i.e., ex vivo/in vitro methods). In one embodiment, test samples are obtained by an in vivo/ex vivo nucleic acid harvesting technique—for example dialysis or functionalized wire.

Test samples may be obtained from patients suspected of having a particular disease or condition, such as cancer. Such a disease or condition can be diagnosed, prognosed, monitored and therapy can be determined based on the methods, systems and kits described herein. Samples may be obtained from humans or from animals, such as a domesticated animal, for example a cow, chicken, pig, horse, rabbit, dog, cat, or goat. Usually, a sample will be derived from a human. In a preferred embodiment, the test sample is a blood sample derived from a human who has previously been diagnosed with cancer and has undergone a first treatment or therapy for cancer. The patient that provides the test sample may have cancer, may have been treated for cancer in the past (e.g., at least 2 weeks before, at least 3 months before, at least 6 months before, at least a year before), may be in complete remission and/or may have a clonal growth (e.g., a tumorous growth such as a nodule, polyp and cyst or lump) that has the potential to transform.

In some embodiments when testing for minimal residual disease or recurrence detection, the test sample from a patient would be cell-free DNA. This cell-free DNA may be taken from a patient at any point after treatment. In some embodiments this cell free DNA may be taken at a point that any remaining ctDNA from a cancer would have been cleared if the cancer were successfully treated. This time point may depend on factors such as the initial amount of ctDNA and the treatment modalities. For methods where all tumor is removed at once such as surgery time points may be after 1 week, 2 weeks, 3 weeks or 4 weeks following treatment with curative intent. Where a treatment may more gradually remove the cancer these time points may be longer such as 1 month or 2 months. As would be apparent, other DNA extracted from alternative sources could also be assessed for the presence or quantity of cancer DNA. Examples include but are not limited to: the cellular fraction of cerebrospinal fluid, the cellular and cell-free fraction of cerebrospinal fluid, stool samples, cells present within urine, biopsy or fine needle aspirate materials.

In some embodiments, the method may also be used to assess for the presence of remaining cancer cells within biopsy or fine needle aspirate materials such as from lymph nodes. As would be apparent such methods would be particularly powerful when the number of tumor cells in a biopsy sample may be at such a low level that it is not practical for histopathological analysis by a pathologist to review enough cells in the biopsy to identify the remaining cancer.

To obtain a blood sample, any technique known in the art may be used, e.g., a syringe or other vacuum suction device. A blood sample can be optionally pre-treated or processed prior to tagging and analysis. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a crosslinking reagent. In addition, plasma may be obtained from the blood sample, and the plasma be used in the subsequent analysis.

When obtaining a sample from a human or an animal (e.g., blood sample), the amount can vary depending upon human or animal size and the condition being screened. In some embodiments, up to 50, 40, 30, 20, 10, 9, 8, 7, 6, 5, 4, 3, 2, or 1 mL of a sample is obtained. In some embodiments, 1-50, 2-40, 3-30, or 4-20 ml of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

A sample may be processed prior to undergoing further analysis. Such processing steps may comprise purification (for example removal of cells and/or debris from the sample), extraction or isolation of a nucleic acid such as DNA. In the case of, for example, blood samples, the DNA may be extracted from the blood sample for analysis. The amount of DNA present in the extracted sample may also be quantified prior to analysis.

In some embodiments, the sample may be obtained from the patient by an in vivo/ex vivo nucleic acid harvesting technique—for example dialysis or functionalised wire.

In particular embodiments, the method comprises a step of obtaining the sample from a patient. In other embodiments, the test sample is simply provided, as a test sample was obtained at a prior point in time. The skilled person is aware of suitable techniques for obtaining, storing, stabilising and/or transporting samples prior to analysis.

The preferred features for the second and subsequent aspects of the disclosure are as provided for the first aspect, mutatis mutandis.

The present invention will now be further explained by reference to a number of non-limiting examples.

### EXAMPLES

### Example 1—Variant Detection and Variant Ranking

Cancer samples are obtained from 10 cancer patients for whom ctDNA MRD analysis is required (2 lung cancer patients, 2 breast cancer patients, 2 colon cancer patients, 2 brain tumor patients and 2 Multiple myeloma patients). For the solid tumor patients an FFPE biopsy sample is obtained. For the Hematological cancer patients a bone marrow aspirate sample is obtained. For each of these patients, either a buffy coat or a buccal swab is also obtained as a matched normal (non-cancer) control. The tumor fraction is first assessed by pathology for all solid tumor samples. For any samples with less than 20% cellularity, microdissection is used to enrich for the tumor containing region. DNA is extracted from each of the samples (e.g. using the QIAamp DNA FFPE Tissue Kit for FFPE samples) then its quality and quantity determined using the TapeStation system (Agilent). The Covaris system is used to fragment DNA in samples with longer molecules including the bone marrow aspirate and buffy coat. A sequencing library is generated from each sample using the KAPA Hyper Prep Library Preparation Kit and the KAPA Library Amplification Kit. Each library is quantified by qPCR, pooled then loaded onto a NovaSeqX (Illumina) for sequencing.

The tumor samples are each sequenced to an average depth of 100×. The non-cancer control samples are sequenced to an average depth of 30×. Sequencing reads are demultiplexed then aligned to the human genome (HG38). SNVs, doublet base substitutions and indels are called genome wide in all samples. Variants with less than 5 supporting reads in the cancer samples are filtered out and variants within repeat regions are also filtered out. Variants detected in the non-cancer control samples are filtered out from the cancer samples as likely germline changes.

The remaining variants are first assessed to determine if they are within 50 bp of another variant that has passed filter. Any that do are grouped together. Variants are then ranked based on the variant allele frequency of each variant (high frequency variants are preferred). In any instance where 2 or more variants have the same frequency (mutant reads/total reads), any variant that has a second variant within 50 bp is selected in preference. If multiple have additional variants, the frequency of the second variant is used to select the top ranking variant. For each patient the top 11,000 variants genome wide are selected.

### Example 2—Twist Based Hybrid Capture Generation

For each patient, Twist Hybrid capture bait sequences (Twist Bioscience) are designed targeting all 11,000 variants. Any variants for whom inefficient capture is predicted are filtered out. Any variants that produce baits which would form secondary structure with the identifier sequence (see below), are also filtered. The top 10,000 remaining variants (by ranking) are selected. For each patient a different 40 bp identifier sequence is added to the 5′ end of the molecule. Each 40 bp sequence has been pre-designed so that it doesn't have significant predicted secondary structure with >99% of the human genome.

The sequences for all 10 patients (100,000 in total) are combined and synthesized using a silicon-based DNA Synthesis platform (Twist) as a pool.

### Example 3—Twist Based Library Preparation, Capture, Sequencing and MRD Calling

10ml of blood is obtained 4 weeks post therapy for all 10 patients in Streck cfDNA BCT. The blood is processed to plasma then cfDNA is extracted using the QIAamp Circulating Nucleic Acid kit (Qiagen).

Digital PCR is performed with a 108 bp assay targeting a region of ribonuclease P/MRP subunit p30 (RPP30) gene using the Biorad QX200 to determine the concentration of the extracted cfDNA.

A minimum of 2,000 copies of cfDNA from each patient (up to a maximum of 40,000) is added to a Twist cfDNA library preparation (if a sample has less than 2,000 copies it would be excluded). Adaptors are ligated to the cfDNA containing molecular barcodes then the DNA is amplified using oligos targeting the ligated adaptors and adding patient specific barcodes.

For each patient the pool of 100,000 oligo bait sequences is combined with an oligo which is complimentary to the patients identifier sequence and has a biotin attached. These oligos are allowed to hybridize. These are then added to standard Twist hybrid capture reaction in place of standard Twist hybrid capture baits in addition to the cfDNA library. Capture is performed for each patient using its unique bait and identifier sequence combination then the libraries are quantified and pooled. The advantage of this invention is that low cost silicon-based DNA Synthesis can be used and the price of the pool can effectively be split between the 10 patients tested. The pooled library is then added to the NovaSeq X (Illumina) sequencer and sequenced to an average of 100,000× per region.

An additional library is produced using buffy coat DNA from each of the blood samples and captured and sequenced in parallel.

### Example 4—MRD Calling

Sequencing reads are demultiplexed and aligned to the Human Genome (HG38). A consensus sequence is made using the molecular barcode sequence on the sequencing read, the start and the end of each sequenced molecule to collapse reads. Consensus families of ≥3 reads are kept. If 1 or more consensus families supporting any of the variants are identifier in the buffy coat, the variant is filter from further analysis as possible CHIP. The remaining variants are called as positive if at least a single family supports the variant (wherein all members of the consensus support the variant). The sample is called as MRD positive if at least 3 different variants are called as positive.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
i. a first sub-population of oligonucleotides that target the plurality of target regions from the first patient, wherein each member of the first sub-population of oligonucleotides comprises a first sequence that is complementary to one of the plurality of target regions from the first patient and an identifier sequence specific to the first patient;
ii. a second sub-population of oligonucleotides, wherein each member of the second sub-population of oligonucleotides comprises a first sequence that is complementary to one of the plurality of target regions from a second patient and an identifier sequence specific to the second patient;
b. contacting a test sample from the first patient with the pool of oligonucleotides;
c. contacting the test sample with oligonucleotides each comprising a sequence that is complementary to the identifier sequence specific to the first patient;
d. selectively sequencing the plurality of target regions from the first patient, optionally identifying the presence or absence of the cancer specific variants.

2. A method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
i. a first sub-population of primer oligonucleotides, wherein each member of the first sub-population of primer oligonucleotides comprises a first sequence that is complementary to one of the target regions from the first patient and an identifier sequence specific to the first patient;
ii. a second sub-population of primer oligonucleotides, wherein each member of the second sub-population of primer oligonucleotides comprises a first sequence that is complementary to one of a plurality of target regions from a second patient and an identifier sequence specific to the second patient;
b. contacting a test sample from the first patient with the pool of oligonucleotides;
c. performing PCR on the test sample comprising the pool of oligonucleotides to produce an amplified test sample;
d. enriching the amplified test sample for amplicons derived from the first patient's target regions by contacting the amplified test sample with patient-specific primer pairs and performing PCR, wherein one or both primers comprises a sequence that is complementary to a identifier sequence specific to the first patient present in the first sub-population of oligonucleotides from step (a);
e. selectively sequencing the enriched sample to determine the sequence of the plurality of target regions from the first patient.

3. The method of claim 2, wherein the primer oligonucleotides are pairs of forward and reverse primers targeting the plurality of target regions.

4. The method of claim 2, wherein each pair of primer oligonucleotides comprises a universal primer and either a forward primer or a reverse primer complementary to one of a plurality of target regions.

5. The method of claim 4, wherein the test sample from the first patient comprises at least one adaptor containing a universal sequence ligated to the test sample, the adaptor comprising a sequence complementary to the universal primer, optionally the adaptor is a Y-stem adaptor.

6. The method of claim 2, wherein the patient-specific primers further comprise sequencing adaptors.

7. The method of claim 6, wherein the sequencing adaptor provides compatibility with a particular sequencing platform, optionally an Illumina sequencing platform.

8. A method for selective sequencing of a plurality of target regions from a first patient, each of the target regions containing, or suspected of containing, one or more cancer specific variants, the method comprising:
a. providing a pool of oligonucleotides, wherein the pool of oligonucleotides comprises:
i. a first sub-population of bait oligonucleotides, wherein the first sub-population of oligonucleotides comprises oligonucleotide baits that target the plurality of target regions from the first patient, wherein each member of the first sub-population of oligonucleotides comprises a first sequence that is complementary to one of the target regions from the first patient and an identifier sequence specific to the first patient;
ii. a second sub-population of bait oligonucleotides, wherein the second sub-population of oligonucleotides comprises oligonucleotide baits wherein each member of the second sub-population of oligonucleotides comprises a first sequence that is complementary to one of a plurality of target regions from a second patient and an identifier sequence specific to the second patient;
b. contacting a test sample from the first patient or an amplified test sample derived from the test sample with the pool of oligonucleotides;
c. contacting the test sample with patient-specific oligonucleotides each comprising a binding moiety and a sequence that is complementary to the identifier sequence specific to the first patient, to produce a tagged test sample;
d. conducting a binding moiety pull down assay on the tagged test sample to obtain an enriched sample enriched for amplicons derived from the first patient's target regions;
e. selectively sequencing the enriched sample to determine the sequence of the plurality of target regions from the first patient.

9. The method of claim 8, further comprising a step of performing PCR on the test sample from the first patient after step (a) to produce an amplified test sample.

10. The method of claim 8, wherein the binding moiety is attached to a support.

11. The method of claim 10, wherein the support is a streptavidin bead.

12. The method of claim 8, wherein the binding moiety is a biotin.

13. The method of claim 8, further comprising making a sequencing library from the test sample.

14-33. (canceled)

34. The method of claim 1, wherein the plurality of target regions from a first patient are genomic target regions.

35. (canceled)

36. The method of claim 1, wherein the number of target regions from the first and/or second patient is from about 100 to about 50,000 target regions.

37-39. (canceled)

40. The method of claim 1, wherein the method comprises, before step (a), identifying the one or more cancer specific variants that are present within the patient's cancer.

41. (canceled)

42. The method of claim 40, wherein the one or more cancer-specific variants comprises 500 to 50,000 cancer specific variants that are present within the patient's cancer.

43. The method of claim 40, wherein identifying one or more specific variants that are present within the patient's cancer comprises identifying 500 or more cancer specific variants that are present within the patient's cancer and selecting a subset of 500 to 50,000 cancer-specific variants.

44-46. (canceled)

47. The method of claim 1, wherein the pool of oligonucleotides further comprises N additional patient-specific sub-populations of oligonucleotides, wherein each member of each patient-specific sub-population of oligonucleotides comprises a first sequence that is complementary to one of a plurality of target regions from that sub-population's patient, and a patient-specific sequence specific to that sub-population's patient, wherein N is from 1 to 50.

48-61. (canceled)

62. The method of claim 1, further comprising combining evidence from each of the plurality of target regions supporting the presence or absence of a cancer-specific variant.

63-66. (canceled)
